SEQUENCE LISTING (30 sequences)

SEQ ID NO: 1 - DNA, 18 bp, Artificial Sequence
  5′ Oligonucleotide primer
gcctaacacatgcaagtc

SEQ ID NO: 2 - DNA, 17 bp, Artificial Sequence
  3′ Oligonucleotide primer
attaccgcggctgctgg

SEQ ID NO: 3 - DNA, 18 bp, Unknown
  From the phylum Spirochaetes
gcctaacacatgcaagtc

SEQ ID NO: 4 - DNA, 18 bp, Unknown
  From the phylum Spirochaetes
gcttaacacatgcaagtc

SEQ ID NO: 5 - DNA, 18 bp, Unknown
  From the phylum Spirochaetes
gtcttaaacatgcaagtc

SEQ ID NO: 6 - DNA, 18 bp, Unknown
  From the phylum Spirochaetes
gtcttaagcatgcaagtc

SEQ ID NO: 7 - DNA, 18 bp, Unknown
  From the phylum Spirochaetes
gttttaagcatgcaagtc

SEQ ID NO: 8 - DNA, 18 bp, Unknown
  From the phylum Spirochaetes
gtcttaagcatgcaagtt

SEQ ID NO: 9 - DNA, 18 bp, Unknown
  From the phylum Spirochaetes
gttttaagcatgcaagtt

SEQ ID NO: 10 - DNA, 18 bp, Artificial Sequence
  Consensus sequence
gtcttaarcatgcaagtc

SEQ ID NO: 11 - DNA, 17 bp, Unknown
  From the phylum Spirochaetes
ccagcagccgcggtaat

SEQ ID NO: 12 - DNA, 17 bp, Unknown
  From the phylum Spirochaetes
ccagcagccgcggtaat

SEQ ID NO: 13 - DNA, 17 bp, Unknown
  From the phylum Spirochaetes
ccagcagccacggtaat

SEQ ID NO: 14 - DNA, 17 bp, Unknown
  From the phylum Spirochaetes
ccagcagccgcggcaat

SEQ ID NO: 15 - DNA, 17 bp, Unknown
  From the phylum Spirochaetes
ccagcagccgcggtaac

SEQ ID NO: 16 - DNA, 18 bp, Unknown
  From the phylum Spirochaetes
ccagcagccgacggtaac

SEQ ID NO: 17 - DNA, 17 bp, Artificial Sequence
  Consensus sequence
ccagcagccgcggtaat

SEQ ID NO: 18 - DNA, 18 bp, Artificial Sequence
  5′ Oligonucleotide primer
gcctaacacatgcaagtc

SEQ ID NO: 19 - DNA, 17 bp, Artificial Sequence
  3′ Oligonucleotide primer
attaccgcggctgctgg

SEQ ID NO: 20 - DNA, 15 bp, Artificial Sequence
  5′ Oligonucleotide primer for Cardiobacterium
gcagcacgagaaagc

SEQ ID NO: 21 - DNA, 15 bp, Artificial Sequence
  3′ Oligonucleotide primer for Cardiobacterium
atcagcgcgaggtct

SEQ ID NO: 22 - DNA, 15 bp, Artificial Sequence
  5′ Oligonucleotide primer for Fusobacterium
ctcttagaccgggac

SEQ ID NO: 23 - DNA, 15 bp, Artificial Sequence
  3′ Oligonucleotide primer for Fusobacterium
gggacgcaaagctct

SEQ ID NO: 24 - DNA, 15 bp, Artificial Sequence
  5′ Oligonucleotide primer for Actinomycetaceae
acgggatctgatggg

SEQ ID NO: 25 - DNA, 15 bp, Artificial Sequence
  3′ Oligonucleotide primer for Actinomycetaceae
cccacaaccaccatg

SEQ ID NO: 26 - DNA, 17 bp, Artificial Sequence
  5′ Oligonucleotide primer for Treponema
cggcaagagagaagctt

SEQ ID NO: 27 - DNA, 17 bp, Artificial Sequence
  3′ Oligonucleotide primer for Treponema
ctctaacagatgcggtc

SEQ ID NO: 28 - DNA, 17 bp, Artificial Sequence
  5′ Oligonucleotide primer for Desulfomicrobium
ccgggaatgagtagagt

SEQ ID NO: 29 - DNA, 17 bp, Artificial Sequence
  3′ Oligonucleotide primer for Desulfomicrobium
catcctttaccgactcc

SEQ ID NO: 30 - DNA, 17 bp, Artificial Sequence
  Reverse complement of 3′ Oligonucleotide primer
ccagcagccgcggtaat

(19) United States
(12) Patent Application Publication
(10) Pub. No.: US 2016/0180018 A1
(43) Pub. Date: Jun. 23, 2016

(54) MOLECULAR AND BIOINFORMATICS METHODS FOR DIRECT SEQUENCING

(71) Applicant: BiSN Laboratory Services Ltd., Runcorn (GB)

(72) Inventors: Andrew Millar, Runcorn (GB); Niels Larsen, 8000 Aarhus C (DK); Paul Broteherton, Runcorn (GB); Heather Allison, Liverpool (GB)

(21) Appl. No.: 14/924,070

(22) Filed: Oct. 27, 2015

(30) Foreign Application Priority Data
Oct. 28, 2014 (GB) 1419167.0
May 29, 2015 (GB) 1509226.5

(51) Int. Cl. G06F 19/14; G06F 19/24; C12Q 1/68; G06F 19/22

(52) U.S. Cl. CPC G06F 19/14; G06F 19/22; G06F 19/24; C12Q 1/689

(57) ABSTRACT

The present invention relates to methods for preparing an isolated biological sample containing at least one of DNA and RNA, such that the DNA and/or RNA is preserved in the sample at ambient temperatures for at least thirty days, the method comprising: contacting the isolated biological sample with a composition comprising a chaotropic agent, and subjecting the contacted sample to microbial cell lysis; and optionally, contacting the lysed biological sample with a slurry of size-selected silicon dioxide to form at least one of DNA-silicon dioxide complexes or RNA-silicon dioxide complexes in the sample; isolating at least one of DNA-silicon dioxide complexes or RNA-silicon dioxide complexes from the sample; and, separating at least one of DNA and RNA from the silicon dioxide and collecting at least one of the DNA and RNA.

The present invention further relates to methods for preparing an isolated biological sample, the method comprising, separating the components in an isolated biological sample according to their size, wherein the components are at least one of DNA and RNA; purifying and isolating SSU rRNA from the biological sample using a composition comprising a ribonuclease inhibitor and a deoxyribonuclease to remove DNA from the sample, reverse transcribing the SSU rRNA into ds cDNA using random primers for SSU rRNA.

The present invention also relates to computer implemented methods comprising, receiving an isolated sample prepared according to the methods of the invention, sequencing the sample, and providing the sequence with a sequence identifier (ID), the sequence comprising a plurality of groups of k-mers, each group of k-mers defining a node in a multi-level hierarchy which defines the relationship between the groups of k-mers; providing each group of k-mers with a respective group identifier (ID), determining the frequency of the k-mers in each group; generating a group signature array for each group of k-mers, each group signature array comprising the k-mers in each group that have the most increased frequency compared with the sibling k-mers; generating a signature map comprising each group signature array and at least one of the identifiers, the identifier of at least one parent group and the identifier of at least one child group; and outputting the signature map to be used to classify the sequence.

This Application claims priority to UK Patent Application UK 1419167.0, filed Oct. 28, 2014 and UK Patent Application UK 1509226.5 filed May 29, 2015, which are incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Jan. 25, 2016, is named bisn_01_rev_ST25.txt and is 6,325 bytes in size.

### DESCRIPTION OF THE INVENTION

The invention relates to methods for isolating, preparing and directly sequencing a biological sample, in particular, methods for isolating, preparing and sequencing 16S or 18S rRNA in an isolated biological sample. The invention further provides for the computer-implemented analysis of sequences in a sample into a collection of classified homologous sequences, useful for example in microbial diagnostics and microbiome analyses.

The invention relates to methods for isolating and preparing a biological sample, such as a sample containing rRNA, mRNA or DNA, for computer-implemented sequencing analysis in combination with computer-implemented analysis of such sequences into a collection of classified homologous sequences.

The present invention has particular utility in the oil and gas fields, in particular, in classifying microbial diversity in biological samples isolated from oil wells.

### BACKGROUND

The biosphere is essentially a diverse consortia of single cellular organisms from all three domains of life, Bacteria, Archaea and Eukarya, most of unknown form and function. Elucidating their true diversity has so far proved difficult. As a consequence, developing microbial diagnostics has also proved challenging.

The use of ribosomal RNA gene sequences as phylogenetic markers revolutionised the study of molecular evolution, phylogeny and ecology in all living organisms. Consequently, our appreciation of microbial diversity on Earth has benefited enormously from Small Sub-Unit ribosomal RNA (SSU rRNA) gene analyses based on the 16S ribosomal RNA gene of Bacteria and Archaea and the 18S ribosomal RNA gene of Eukarya, providing a phylogenetic framework for the classification and assessment of microbial diversity in any given environment without the requirement for isolation and cultivation.

Because as many as 99.9% of the microorganisms in a particular environment are intractable to current cultivation strategies, the analysis of SSU rRNA gene sequences provides the primary tool to address the “great plate count anomaly”.

Current methods for assessing biological diversity use DNA or RNA gene markers in combination with high-throughput DNA and rRNA sequencing.

Those studies have focussed on PCR amplification and sequencing of microbial rRNA genes. Consequently, today's universal phylogenetic tree contains many microbial lineages that are delimited only by uncultivated microorganisms, and this number continues to increase. For example, in 1987 there were 12 bacterial phylogenetic divisions based entirely on cultured isolates, but by 2004 there were ˜80 divisions (26 based on cultured isolates and ˜54 on DNA sequence data only).

However, current methods are unable to handle the large amount of sequence data produced from such high-throughput DNA and rRNA sequencing. This has led to difficulties in classifying the resulting sequence data in a meaningful manner, particularly in terms of data accuracy and the speed of production of the data.

Additionally, sample preparation issues compound the quality of the sample used in the currently sequence handling and sequencing methods. There are inherent biases in current sample preparation approaches for such high-throughput sequencing, which can add another level of complexity to methods of sequencing the samples and classifying the resulting sequence data in a meaningful manner.

Current sample preparation and nucleic acids extraction methods also suffer from quality issues caused by degradation of DNA, rRNA and mRNA in samples; particularly during the time which elapses between sample collection, nucleic acid extraction and the ultimate ‘fixation’ of DNA and/or RNA (in the form of cDNA) sequences in Next Generation Sequencing (NGS) libraries. Also, the samples can suffer from quality issues where impurities, such as production chemicals, natural ions, biomolecules and qPCR assay inhibitors, can inhibit DNA and RNA extraction.

Current sample preparation methods, particularly those employed in the oil and gas fields, often utilise bulk water or filter-based methods. For example, bulk water methods typically involve simply collecting fluid in a vessel of some description and transporting the vessel back to the analytical laboratory (either at ambient temperatures or chilled to 4° C.). Filter-based methods typically involve passing a fluid sample through a filter and collecting any prokaryotic cells on the filter membrane. The filtered prokaryotic cells are then suspended in an RNA preserver, such as RNAlater®. (The exact formulation of RNAlater® is proprietary; although it is believed to be based on ammonium sulphate.) However, a key consideration in sampling, often overlooked particularly in the oil and gas industries, is extraction efficiency. Prior art methods, such as filter-based methods, often result in significant fragmentation and degradation of DNA and/or rRNA/mRNA in samples; particularly during prolonged storage and/or transportation.

One way to avoid such DNA—and particularly RNA—fragmentation and degradation would be to perform sequencing directly at the point of sample collection. However, the technologies behing portable sequencing devices is not yet at a stage to make this realistic in the oil and gas industries. Laboratory equipment and freezers are not generally available in the field to preserve and store isolated biological samples. Therefore, by the time biological samples isolated using current bulk water and filter-based methods are transported to a laboratory for processing, nucleic acids extraction, analyses and sequencing, much of the DNA—and especially the RNA—in the sample may have been significantly fragmented and degraded. Therefore there exists a pressing need for improved sample processing methods that can preserve both DNA and RNA indefinitely, particularly in the oil and gas fields or in other situations where rapid sample processing and sequencing is not possible at the point of sample collection.

The use of artificial amplification of sequences, such PCR approaches, is the paradigm in high-throughput DNA and rRNA sequencing. For example, the PCR amplification and sequencing of phylogenetic markers, primarily SSU rRNA genes, is the paradigm for defining the taxonomic composition of microbiomes.

PCR-associated biases stem from two effectors: 1) different genomic DNA templates exhibit different PCR amplification efficiencies impinging on both detection of taxa and estimates of their relative abundance; 2) PCR primer sets can only be designed to target ‘known diversity’ as represented in public databases, and the introduction of relaxed specificity and degeneracy in primer design provides only a very limited expansion of that. It has been estimated that certain ‘universal’ PCR primer sets miss 50% of the microbial rRNA gene diversity. Consequently, rRNA gene inventories derived from PCR amplicons miss a proportion of unexplored diversity and provide potentially misleading estimates of relative abundance, especially if the unidentified taxa are present in significant numbers. Furthermore, most molecular microbial ecology studies focus on only one of the three microbial domains, usually Bacterial 16S rRNA genes.

One way to attempt to overcome the biases would be to sequence the entire rRNA (direct ‘total RNA metatranscriptome’ sequencing). However, this would take a long time and the large volume of sequence data produced would be too complex to analyse with currently available sequencing platforms. Additionally, total RNA metatranscriptomes comprise mRNA, and both small- and large-subunit rRNA, with only ca. 40% of the sequence output representing SSU rRNA gene sequences. Therefore, this is not a viable method for analysing data to provide a collection of classified homologous sequences, for example in taxonomic studies.

Thus, there exists a need for computer-implemented methods for handling and analysing large quantities of sequence data in a meaningful way. There also exists a need for improved sample preparation methods to improve the quality of the sample for use in sequencing analysis.

The object of the present invention is to provide an improved (faster and more accurate) computer-implemented method for sequencing a sample, and handling and analysing large quantities of sequence data in a meaningful way.

A further object of the present invention is to provide an improved sample preparation method, which can be used in combination with the aforementioned computer-implemented method. In particular, an object of the present invention is to provide a fast and accurate method for sample preparation and the classification of sequences in samples into a large collection of classified homologous sequences. A further object of the present invention is to provide a sample preparation method which minimises the degradation of DNA and RNA and optimises the amount of DNA and RNA extracted from the sample. Properly processed samples can be stored at ambient temperatures for transportation or storage until they can be fully extracted for DNA/RNA. This sample processing methodology can be used in combination with the aforementioned computer-implemented method. The present invention has particular utility in the oil and gas fields or in situations where sample processing and sequencing is not possible at the point of sample collection.

### SUMMARY OF THE INVENTION

An embodiment of the present invention seeks to provide a high throughput method for biological sample isolation, preparation and sequencing. The present invention has particular utility in the oil and gas fields, in particular, in classifying microbial diversity in biological samples isolated from oil wells.

According to the present invention, there is provided a method for sample preparation.

According to aspects of the present invention, there is provided a method for preparing an isolated biological sample containing at least one of DNA and RNA, such that the DNA and/or RNA is preserved in the sample at ambient temperatures for at least days, the method comprising: contacting the isolated biological sample with a composition comprising a chaotropic agent, and subjecting the contacted sample to microbial cell lysis. Advantageously, the method further comprises subjecting the isolated biological sample to an activated charcoal treatment step. Preferably, the isolated biological sample is subjected to the activated charcoal treatment step before, simultaneously with or subsequent to contacting the isolated biological sample with a composition comprising a chaotropic agent. In some aspects, the isolated biological sample is subjected to the activated charcoal treatment step before, simultaneously with or subsequent to subjecting the contacted sample to microbial cell lysis.

Advantageously, both DNA and RNA are isolated simultaneously in the methods of the present invention.

Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate. Advantageously, the composition further comprises a solvent.

Preferably, the solvent is alcohol-based; More preferably, the solvent is selected from isopropanol (IPA or propan-2-ol) or ethanol or combinations thereof.

Preferably, the contacting composition comprises guanidine thiocyanate (GTC) solution and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

Advantageously, the microbial cell lysis is mechanical lysis. Preferably, the contacted sample is passed through a chamber containing beads (such as glass beads) which are spun via a motor means to cause mechanical lysis of the contact sample. In some aspects, a micro-lyser device can be employed to perform mechanical lysis. Preferably, a battery-powered micro-lyser device is employed.

Conveniently, the microbial cell lysis can be performed by the contacting composition comprising a chaotropic agent. In additional aspects, the microbial cell lysis is performed by the contacting composition comprising a chaotropic agent and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Preferably, the isolated biological sample is from an oil well.

Further aspects of the present invention relate to use of a composition comprising or consisting of a chaotropic agent in combination with a means for performing microbial cell lysis, for preserving at least one of DNA and RNA in an isolated biological sample containing at least one of DNA and RNA, at ambient temperatures for at least thirty days.

Advantageously, the use further comprises subjecting the isolated biological sample to an activated charcoal treatment step. Preferably, the isolated biological sample is subjected to the activated charcoal treatment step before, simultaneously with or subsequent to contacting the isolated biological sample with a composition comprising a chaotropic agent. In some aspects, the isolated biological sample is subjected to the activated charcoal treatment step before, simultaneously with or subsequent to subjecting the contacted sample to microbial cell lysis.

Advantageously, both DNA and RNA are isolated. Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

Advantageously, the composition further comprises a solvent.

Preferably, the solvent is selected from isopropanol (IPA or propan-2-ol) or ethanol or combinations thereof.

Preferably, the contacting composition comprises guanidine thiocyanate (GTC) and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Advantageously, the microbial cell lysis is mechanical lysis. Preferably, the contacted sample is passed through a chamber containing beads (such as glass beads) which are spun via a motor means to cause mechanical lysis of the contact sample. In some aspects, the means for performing microbial cell lysis can be a micro-lyser device. Preferably, the means for performing microbial cell lysis is a battery-operated micro-lyser device.

Conveniently, the microbial cell lysis can be performed by the composition comprising a chaotropic agent. In additional aspects, the means for performing microbial cell lysis can be a composition comprising the chaotropic agent and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

Preferably, the isolated biological sample is from an oil well.

Another aspect of the present invention relates to a kit comprising a composition comprising or consisting of a chaotropic agent in combination with a means for performing microbial cell lysis.

Advantageously, the kit can further include instructions for preserving at least one of DNA and RNA in an isolated biological sample containing at least one of DNA and RNA, at ambient temperatures for at least thirty days. Preferably, the isolated biological sample is from an oil well. Advantageously, the instructions are for isolation of both DNA and RNA.

Advantageously, the kit further includes instructions for subjecting the isolated biological sample to an activated charcoal treatment step. Preferably, the instructions provide for subjecting the isolated biological sample to the activated charcoal treatment step before, simultaneously with or subsequent to contacting the isolated biological sample with the composition comprising or consisting of a chaotropic agent. In some aspects, the instructions provide for subjecting the isolated biological sample to the activated charcoal treatment step before, simultaneously with or subsequent to subjecting the contacted sample to microbial cell lysis.

Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

Advantageously, the composition further comprises a solvent.

Preferably, the solvent is selected from isopropanol (IPA or propan-2-ol) or ethanol or combinations thereof.

Preferably, the contacting composition comprises guanidine thiocyanate (GTC) and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Advantageously, the microbial cell lysis is mechanical lysis. Preferably, the contacted sample is passed through a chamber containing beads (such as glass beads) which are spun via a motor means to cause mechanical lysis of the contact sample. In some aspects, the means for performing microbial cell lysis can be a micro-lyser device. Preferably, the means for performing microbial cell lysis is a battery-operated micro-lyser device.

Conveniently, the microbial cell lysis can be performed by the composition comprising a chaotropic agent. In additional aspects, the means for performing microbial cell lysis can be a composition comprising the chaotropic agent and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

According to another aspect of the present invention, there is provided a method for preparing an isolated biological sample containing at least one of DNA and RNA, the method comprising:
(i) contacting the isolated biological sample with a composition comprising a chaotropic agent, and subjecting the contacted sample to microbial cell lysis;
(ii) contacting the lysed biological sample with a slurry of size-selected silicon dioxide to form at least one of DNA-silicon dioxide complexes or RNA-silicon dioxide complexes in the sample;
(iii) isolating at least one of DNA-silicon dioxide complexes or RNA-silicon dioxide complexes from the sample (or both); and
(iv) separating at least one of DNA and RNA from the silicon dioxide and collecting at least one of the DNA and RNA.

Advantageously, the method further comprises subjecting the isolated biological sample to an activated charcoal treatment step. Preferably, the isolated biological sample is subjected to the activated charcoal treatment step before, simultaneously with or subsequent to contacting the isolated biological sample with a composition comprising a chaotropic agent. In some aspects, the isolated biological sample is subjected to the activated charcoal treatment step before, simultaneously with or subsequent to subjecting the contacted sample to microbial cell lysis.

Advantageously, both DNA and RNA are isolated in the methods of the present invention.

Preferably, the silicon dioxide is in the form of size-selected silicon dioxide beads. More preferably, the size-selected silicon dioxide beads are in a solvent, such as water.

Preferably, the pH of the lysed biological sample in the final binding buffer is less than 7.0 (to maximise the binding of DNA and RNA to silica).

Conveniently, the lysed biological sample is contacted with a solvent before contacting the sample with a slurry of size-selected silicon dioxide. Preferably, the solvent is selected from isopropanol (IPA/propan-2-ol) or ethanol or combinations thereof. More preferably, when guanidine thiocyanate (GTC) is used as the chaotropic agent in step (i) (i.e. in the contacting composition, the solvent is isopropanol (IPA/propan-2-ol).

Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

Advantageously, the composition comprising a chaotropic agent (contacting composition) further comprises a solvent. Preferably, the solvent is selected from isopropanol (IPA/propan-2-ol) or ethanol or combinations thereof.

Preferably, the contacting composition comprises guanidine thiocyanate (GTC) and a solvent selected from isopropanol (IPA/propan-2-ol) or ethanol or combinations thereof. The contacting composition may further comprise at least one additional ingredient selected from a buffer, sodium chloride or a detergent (such as Triton X-100) and combinations thereof.

Advantageously, the microbial cell lysis is mechanical lysis. Preferably, the contacted sample is passed through a chamber containing beads (such as glass beads) which are spun via a motor means to cause mechanical lysis of the contact sample. In some aspects, a micro-lyser device can be employed to perform mechanical lysis. Preferably, a battery-powered micro-lyser device is employed.

Conveniently, the microbial cell lysis can be performed by the contacting composition comprising a chaotropic agent. In additional aspects, the microbial cell lysis is performed by the contacting composition comprising a chaotropic agent and a solvent selected from isopropanol (IPA/propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Preferably, the isolated biological sample is from an oil well.

Conveniently, the method of isolating at least one of DNA-silicon dioxide complexes and/or RNA-silicon dioxide complexes from the sample comprises:
(i) rotating and centrifuging the sample to produce a pellet containing the DNA-silicon dioxide complexes and/or RNA-silicon dioxide complexes;
(ii) washing the pelleted beads in 70-80% ethanol solution to remove binding buffer components from the DNA-silicon dioxide complexes and/or RNA-silicon dioxide complexes; and
(iii) resuspending the DNA-silicon dioxide complexes and/or RNA-silicon dioxide complexes in a buffer.

Another aspect of the present invention relates to a kit comprising a composition comprising or consisting of a chaotropic agent in combination with a means for performing microbial cell lysis and a slurry of size selected silicon dioxide.

Preferably, the silicon dioxide is in the form of size-selected silicon dioxide beads. More preferably, the size-selected silicon dioxide beads are in a solution, such as water.

Advantageously, the kit can further include instructions for preserving and isolating at least one of DNA and RNA from an isolated biological sample containing at least one of DNA and RNA, at ambient temperatures for at least thirty days. Preferably, the isolated biological sample is from an oil well. Advantageously, the instructions are for isolation of both DNA and RNA.

Advantageously, the kit further includes instructions for subjecting the isolated biological sample to an activated charcoal treatment step. Preferably, the instructions provide for subjecting the isolated biological sample to the activated charcoal treatment step before, simultaneously with or subsequent to contacting the isolated biological sample with the composition comprising or consisting of a chaotropic agent. In some aspects, the instructions provide for subjecting the isolated biological sample to the activated charcoal treatment step before, simultaneously with or subsequent to subjecting the contacted sample to microbial cell lysis. In some aspects, the instructions provide for subjecting the isolated biological sample to the activated charcoal treatment before contacting the lysed biological sample with a slurry of size-selected silicon dioxide.

Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

Advantageously, the composition further comprises a solvent.

Preferably, the solvent is selected from isopropanol (IPA/propan-2-ol) or ethanol or combinations thereof.

Preferably, the contacting composition comprises guanidine thiocyanate (GTC) and a solvent selected from isopropanol (IPA/propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Advantageously, the microbial cell lysis is mechanical lysis. Preferably, the contacted sample is passed through a chamber containing beads (such as glass beads) which are spun via a motor means to cause mechanical lysis of the contact sample. In some aspects, the means for performing microbial cell lysis can be a micro-lyser device. Preferably, the means for performing microbial cell lysis is a battery-operated micro-lyser device.

Conveniently, the microbial cell lysis can be performed by the composition comprising a chaotropic agent. In additional aspects, the means for performing microbial cell lysis can be a composition comprising the chaotropic agent and a solvent selected from isopropanol (IPA/propan-2-ol) or ethanol or combinations thereof. The composition may further comprise at least one additional ingredient selected from a buffer, sodium chloride or a detergent (such as Triton X-100) and combinations thereof.

According to an aspect of the present invention, there is provided a method for preparing an isolated biological sample, the method comprising: separating the components in an isolated biological sample according to their size, wherein the components are at least one of DNA and RNA; purifying and isolating SSU rRNA from the biological sample using a composition comprising a ribonuclease inhibitor and a deoxyribonuclease to remove DNA from the sample, reverse transcribing the SSU rRNA into ds cDNA using random primers for SSU rRNA. Preferably, during the step of reverse transcribing SSU rRNA into ds cDNA using random primers for SSU rRNA, no amplification occurs.

Conveniently, during the step of reverse transcribing SSU rRNA into ds cDNA using random primers for SSU rRNA, no PCR amplification occurs.

Advantageously, the ds cDNA is amplified by artificial amplification.

Preferably, the artificial amplification is PCR amplification.

In one embodiment, the method does not comprise a step of amplification of the isolated sample.

In another embodiment, the method does not comprise a step of PCR amplification of the isolated sample.

Preferably, the isolated biological sample is from an oil well.

According to another aspect of the present invention, there is provided a method for preparing and sequencing an isolated biological sample, the method comprising: separating the components in an isolated biological sample according to their size, wherein the components are at least one of DNA and RNA; purifying and isolating the desired component or components from the biological sample; wherein,
(a) when the desired component is RNA, Small Sub-Unit ribosomal RNA (SSU rRNA) is isolated and purified using a composition comprising a ribonuclease inhibitor and a deoxyribonuclease to remove DNA from the sample, which SSU rRNA is then reverse transcribed into ds cDNA; or
(b) when the desired component is RNA, SSU rRNA is isolated and purified followed by artificial amplification; or
(c) when the desired component is DNA, DNA is isolated and purified followed by artificial amplification; and further comprising:

sequencing the sample, providing the sequence with a sequence identifier (ID), the sequence comprising a plurality of groups of k-mers, each group of k-mers defining a node in a multi-level hierarchy which defines the relationship between the groups of k-mers; providing each group of k-mers with a respective group identifier (ID), determining the frequency of the k-mers in each group; generating a group signature array for each group of k-mers, each group signature array comprising the k-mers in each group that have the most increased frequency compared with the sibling k-mers; generating a signature map comprising each group signature array and at least one of the identifiers, the identifier of at least one parent group and the identifier of at least one child group; and outputting the signature map to be used to classify the sequence.

Preferably, in part (b), the artificial amplification method is RT-PCR amplification.

Conveniently, in part (c), the artificial amplification method is PCR amplification.

Advantageously, in part (a), during the step of reverse transcribing SSU rRNA into ds cDNA using random primers for SSU rRNA, no amplification occurs.

Preferably, in part (a), during the step of reverse transcribing SSU rRNA into ds cDNA using random primers for SSU rRNA, no PCR amplification occurs.

Conveniently, the ds cDNA is amplified by artificial amplification.

Advantageously, the artificial amplification is PCR amplification.

Preferably, in part (a), the method does not comprise a step of amplification of the isolated sample.

Conveniently, in part (a), wherein the method does not comprise a step of PCR amplification of the isolated sample.

Advantageously, the isolated biological sample is from an oil well.

In aspects of the invention, the isolated biological sample can be prepared for sequencing using a combination of the aforementioned methods.

For example, in one aspect of the present invention, there is provided a method for preparing an isolated biological sample containing at least one of DNA and RNA, for use in sequencing, the method comprising:
(i) contacting the isolated biological sample with a composition comprising a chaotropic agent, and subjecting the contacted sample to microbial cell lysis;
(ii) contacting the lysed biological sample with a slurry of size-selected silicon dioxide to form at least one of DNA-silicon dioxide complexes or RNA-silicon dioxide complexes in the sample;
(iii) isolating at least one of DNA-silicon dioxide complexes or RNA-silicon dioxide complexes from the sample; and
(iv) separating at least one of DNA and RNA from the silicon dioxide and collecting at least one of the DNA and RNA; and
(v) separating at least one of DNA and RNA in the collected sample according to their size; purifying and isolating SSU rRNA from the biological sample using a composition comprising a ribonuclease inhibitor and a deoxyribonuclease to remove DNA from the sample, reverse transcribing the SSU rRNA into ds cDNA using random primers for SSU rRNA.

Advantageously, the method further comprises subjecting the isolated biological sample to an activated charcoal treatment step. Preferably, the isolated biological sample is subjected to the activated charcoal treatment step before, simultaneously with or subsequent to contacting the isolated biological sample with a composition comprising a chaotropic agent. In some aspects, the isolated biological sample is subjected to the activated charcoal treatment step before, simultaneously with or subsequent to subjecting the contacted sample to microbial cell lysis. In some aspects, the isolated biological sample is subjected to the activated charcoal treatment before contacting the lysed biological sample with a slurry of size-selected silicon dioxide.

Preferably, the silicon dioxide is in the form of size-selected silicon dioxide beads. More preferably, the size-selected silicon dioxide beads are in a solution, such as water.

Conveniently, the lysed biological sample is contacted with a solvent before contacting the sample with a slurry of size-selected silicon dioxide. Preferably, the solvent is selected from isopropanol (IPA/propan-2-ol) or ethanol or combinations thereof. More preferably, when guanidine thiocyanate (GTC) is used as the chaotropic agent in step (i) (i.e. in the contacting composition, the solvent is isopropanol (IPA/propan-2-ol).

Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

Advantageously, the composition comprising a chaotropic agent (contacting composition) further comprises a solvent. Preferably, the solvent is selected from isopropanol (IPA/propan-2-ol) or ethanol or combinations thereof.

Preferably, the contacting composition comprises guanidine thiocyanate (GTC) and a solvent selected from isopropanol (IPA/propan-2-ol) or ethanol or combinations thereof. The contacting composition may further comprise at least one additional ingredient selected from a buffer, sodium chloride or a detergent (such as Triton X-100) and combinations thereof.

Advantageously, the microbial cell lysis is mechanical lysis. Preferably, the contacted sample is passed through a chamber containing beads (such as glass beads) which are spun via a motor means to cause mechanical lysis of the contact sample. In some aspects, a micro-lyser device can be employed to perform mechanical lysis. Preferably, a battery-powered micro-lyser device is employed.

Conveniently, the microbial cell lysis can be performed by the contacting composition comprising a chaotropic agent. In additional aspects, the microbial cell lysis is performed by the contacting composition comprising a chaotropic agent and a solvent selected from isopropanol (IPA/propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Preferably, the isolated biological sample is from an oil well.

Conveniently, the method of isolating at least one of DNA-silicon dioxide complexes or RNA-silicon dioxide complexes from the sample comprises:
(i) rotating and centrifuging the sample to produce a pellet containing the DNA-silicon dioxide complexes or RNA-silicon dioxide complexes;
(ii) washing the pelleted beads in 70-80% ethanol solution to remove binding buffer components from the DNA-silicon dioxide complexes and/or RNA-silicon dioxide complexes; and
(iii) resuspending the DNA-silicon dioxide complexes or RNA-silicon dioxide complexes in a buffer.

Preferably, during the step of reverse transcribing SSU rRNA into ds cDNA using random primers for SSU rRNA, no amplification occurs.

Conveniently, during the step of reverse transcribing SSU rRNA into ds cDNA using random primers for SSU rRNA, no PCR amplification occurs.

Advantageously, the ds cDNA is amplified by artificial amplification.

Preferably, the artificial amplification is PCR amplification.

In one embodiment, the method does not comprise a step of amplification of the isolated sample.

In another embodiment, the method does not comprise a step of PCR amplification of the isolated sample.

According to another aspect, there is provided a method for preparing and sequencing an isolated biological sample, the method comprising:
(i) contacting the isolated biological sample with a composition comprising a chaotropic agent, and subjecting the contacted sample to microbial cell lysis;
(ii) contacting the lysed biological sample with a slurry of size-selected silicon dioxide to form at least one of DNA-silicon dioxide complexes or RNA-silicon dioxide complexes in the sample;
(iii) isolating at least one of DNA-silicon dioxide complexes or RNA-silicon dioxide complexes from the sample; and
(iv) separating at least one of DNA and RNA from the silicon dioxide and collecting at least one of the DNA and RNA; and further comprising:
(v) separating the at least one of DNA and RNA in the collected sample according to their size; purifying and isolating the desired component or components from the biological sample; wherein,
   (a) when the desired component is RNA, Small Sub-Unit ribosomal RNA (SSU rRNA) is isolated and purified using a composition comprising a ribonuclease inhibitor and a deoxyribonuclease to remove DNA from the sample, which SSU rRNA is then reverse transcribed into ds cDNA; or
   (b) when the desired component is RNA, SSU rRNA is isolated and purified followed by artificial amplification; or
   (c) when the desired component is DNA, DNA is isolated and purified followed by artificial amplification; and further comprising:

sequencing the sample, providing the sequence with a sequence identifier (ID), the sequence comprising a plurality of groups of k-mers, each group of k-mers defining a node in a multi-level hierarchy which defines the relationship between the groups of k-mers; providing each group of k-mers with a respective group identifier (ID), determining the frequency of the k-mers in each group; generating a group signature array for each group of k-mers, each group signature array comprising the k-mers in each group that have the most increased frequency compared with the sibling k-mers; generating a signature map comprising each group signature array and at least one of the identifiers, the identifier of at least one parent group and the identifier of at least one child group; and outputting the signature map to be used to classify the sequence.

Advantageously, the method further comprises subjecting the isolated biological sample to an activated charcoal treatment step. Preferably, the isolated biological sample is subjected to the activated charcoal treatment step before, simultaneously with or subsequent to contacting the isolated biological sample with a composition comprising a chaotropic agent. In some aspects, the isolated biological sample is subjected to the activated charcoal treatment step before, simultaneously with or subsequent to subjecting the contacted sample to microbial cell lysis. In some aspects, the isolated biological sample is subjected to the activated charcoal treatment before contacting the lysed biological sample with a slurry of size-selected silicon dioxide.

Preferably, the silicon dioxide is in the form of size-selected silicon dioxide beads. More preferably, the size-selected silicon dioxide beads are in a solution, such as water.

Conveniently, the lysed biological sample is contacted with a solvent before contacting the sample with a slurry of size-selected silicon dioxide. Preferably, the solvent is selected from isopropanol (IPA or propan-2-ol) or ethanol or combinations thereof. More preferably, when guanidine thiocyanate (GTC) is used as the chaotropic agent in step (i) (i.e. in the contacting composition, the solvent is isopropanol (IPA or propan-2-ol).

Conveniently, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

Advantageously, the composition comprising a chaotropic agent (contacting composition) further comprises a solvent. Preferably, the solvent is selected from isopropanol (IPA or propan-2-ol) or ethanol or combinations thereof.

Preferably, the contacting composition comprises guanidine thiocyanate (GTC) and a solvent selected from isopropanol (IPA or propan-2-ol) or ethanol or combinations thereof. The contacting composition may further comprise at least one additional ingredient selected from a buffer, sodium chloride or a detergent (such as Triton X-100) and combinations thereof.

Advantageously, the microbial cell lysis is mechanical lysis. Preferably, the contacted sample is passed through a chamber containing beads (such as glass beads) which are spun via a motor means to cause mechanical lysis of the contact sample. In some aspects, a micro-lyser device can be employed to perform mechanical lysis. Preferably, a battery-powered micro-lyser device is employed.

Conveniently, the microbial cell lysis can be performed by the contacting composition comprising a chaotropic agent. In additional aspects, the microbial cell lysis is performed by the contacting composition comprising a chaotropic agent and a solvent selected from isopropanol (IPA or propan-2-ol) or ethanol or combinations thereof. The composition may further comprise at least one additional ingredient selected from a buffer, sodium chloride or a detergent (such as Triton X-100) and combinations thereof.

Preferably, the DNA and/or RNA can be preserved in the sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

Preferably, the isolated biological sample is from an oil well.

Conveniently, the method of isolating at least one of DNA-silicon dioxide complexes or RNA-silicon dioxide complexes from the sample comprises:
(i) rotating and centrifuging the sample to produce a pellet containing the DNA-silicon dioxide complexes or RNA-silicon dioxide complexes;
(ii) washing the pelleted beads in 70-80% ethanol solution to remove binding buffer components from the DNA-silicon dioxide complexes and/or RNA-silicon dioxide complexes; and
(iii) resuspending the DNA-silicon dioxide complexes or RNA-silicon dioxide complexes in a buffer.

Preferably, in part (v)(b), the artificial amplification method is RT-PCR amplification.

Conveniently, in part (v)(c), the artificial amplification method is PCR amplification.

Advantageously, in part (v)(a), during the step of reverse transcribing SSU rRNA into ds cDNA using random primers for SSU rRNA, no amplification occurs.

Preferably, in part (v)(a), during the step of reverse transcribing SSU rRNA into ds cDNA using random primers for SSU rRNA, no PCR amplification occurs.

Conveniently, the ds cDNA is amplified by artificial amplification.

Advantageously, the artificial amplification is PCR amplification.

Preferably, in part (v)(a), the method does not comprise a step of amplification of the isolated sample.

Conveniently, in part (v)(a), wherein the method does not comprise a step of PCR amplification of the isolated sample.

According to another aspect of the present invention, there is provided a computer implemented method comprising: receiving an isolated sample prepared according to the method of claim 1, sequencing the sample, and providing the sequence with a sequence identifier (ID), the sequence comprising a plurality of groups of k-mers, each group of k-mers defining a node in a multi-level hierarchy which defines the relationship between the groups of k-mers; providing each group of k-mers with a respective group identifier (ID), determining the frequency of the k-mers in each group; generating a group signature array for each group of k-mers, each group signature array comprising the k-mers in each group that have the most increased frequency compared with the sibling k-mers; generating a signature map comprising each group signature array and at least one of the identifiers, the identifier of at least one parent group and the identifier of at least one child group; and outputting the signature map to be used to classify the sequence.

According to another aspect of the present invention, there is provided a computer implemented method comprising: receiving an isolated 16s rRNA sequence, sequencing the sample, and providing the sequence with a sequence identifier (ID), the sequence comprising a plurality of groups of k-mers, each group of k-mers defining a node in a multi-level hierarchy which defines the relationship between the groups of k-mers; providing each group of k-mers with a respective group identifier (ID), determining the frequency of the k-mers in each group; generating a group signature array for each group of k-mers, each group signature array comprising the k-mers in each group that have the most increased frequency compared with the sibling k-mers; generating a signature map comprising each group signature array and at least one of the identifiers, the identifier of at least one parent group and the identifier of at least one child group; and outputting the signature map to be used to classify the sequence.

Preferably, the method comprises receiving a plurality of 16s rRNA sequences, providing each sequence with a respective sequence identifier and indexing the sequences using their identifiers as a key.

Conveniently, the method further comprises: generating a group signature array for each group of k-mers, each group signature array comprising the k-mers in each group that have the most increased frequency compared with the sibling k-mers.

Advantageously, the method further comprises: converting the value of each group into a string; and storing the string for each group with the respective group identifier.

Preferably, if there are more than three sequences associated with a group, the method comprises clustering the sequences into one or more sub-groups, each with a respective sub-group identifier.

Conveniently, the step of generating a group signature array comprises depth first recursive processing of the groups in the hierarchy.

Advantageously, the depth first recursive processing comprises, processing a parent group and each child group of the parent group by: scaling each child group signature array by a maximum value (N) and adding the scaled child group signature array to the parent group signature array.

Preferably, if there are sequences among the child groups then the method comprises converting the sequences to the same signature array format as the parent group signature array to generate a child sum array for each child and adding the converted sequences to one another to form a children sum array.

Conveniently, the method further comprises generating a signature group array for each child by: subtracting the child sum array from the children sum array to produce a siblings sum array; filling the group signature array with the child k-mers in each group with a higher frequency than k-mers in at least one sibling group up to a predetermined frequency value; and scaling the group signature array by the maximum value (N).

Advantageously, the method further comprises classifying a sequence by comparing the sequence to a first child group signature array and comparing the sequence to at least one further child group signature array until no better match can be identified between the sequence and a child group signature array.

Preferably, the method further comprises clustering sequences with a similarity above a predetermined level and mapping the cluster of sequences to the signature map.

According to another aspect of the present invention, there is provided a tangible computer readable medium storing instructions which, when executed by a computing device, cause the computing device to perform the method of any one of claims 19 to 30 defined hereinafter.

According to another aspect of the present invention, there is provided a system for sequencing a biological sample, the system comprising: a processor; and a memory storing computer readable instructions which, when executed by the processor, cause the processor to perform the method of any one of claims 9 to 30 defined hereinafter.

### DESCRIPTION OF DRAWINGS

Non-limiting embodiments of the present invention will now further be described by way of reference to the figures, in which:

FIG. 1 exemplifies the abundance of the sequenced reverse-transcribed microbial SSU rRNA molecules from the canine oral cavity. Domain-level (A) and phylum level classification and abundance of Archaea (B), Eukarya (C) and Bacteria (D) using the SILVA SSU rRNA database (version 108). Only phyla with a relative abundance >0.1% have been included.

FIG. 2 provides a comparison of phylum level classification of PCR amplicon and RT-SSU rRNA sequence reads derived from canine plaque samples.

FIG. 3 provides a comparison of the number of taxa detected at each phylogenetic rank in a 16S rRNA gene amplicon library (PCR) and the sequenced RT-SSU rRNA library (RT-rRNA) generated from canine plaque samples. The datasets were compared using the command line RDP library compare function. Blue and red bars denote the number of taxa detected at each phylogenetic level in the RT-SSU rRNA and 16S rRNA gene amplicon dataset, respectively. Green bars indicate the number of taxa that were common to both datasets.

FIG. 4 exemplifies primer mismatch ratios for phyla detected using an RT-SSU rRNA approach. RT-SSU rRNA sequences containing regions corresponding to the forward and reverse PCR primer sites used to generate the PCR amplicon library in this study were aligned against their closest database match and the number of insertions, deletions (indels) or mutations within the primer binding site recorded. Primer mismatch ratios were calculated by dividing the total number of sequence reads containing the primer-binding site by the total number of indels and mutations recorded within the primer binding sites of those sequences.

FIG. 5 exemplifies quantitative PCR analysis of amplification efficiencies of an artificial microbial community comprising five cloned 16S rRNA genes of canine oral bacteria. The artificial community was generated by mixing ratios of known gene copy number (A9, C10, F10, E3 and E9 in the ratio of 1:3:8:2:10, respectively), followed by 10, 20 or 30 cycles of PCR amplification using the universal bacterial primer set applied in this study. The resulting community PCR amplicons were subjected to qPCR analysis using clone-specific primer sets to determine the relative ratios of each clone in the final amplification mix. Error bars represent the standard error of the mean from 3 independent biological replicates. Data from each biological replicate was obtained from three experimental replicates.

FIG. 6 is a schematic diagram of an embodiment of the invention which comprises a computing device.

FIG. 7 shows input-versus-output over orders-of-magnitude differences in prokaryotic genomic DNA recovery from an oil and gas industry low biomass environmental sample—using the sample isolation methods of the present invention.

### DEFINITIONS

The terms in quotes are used below and have the following meanings:

“16s rRNA” refers to 16s ribosomal RNA. 16s rRNA is a component of the 30s small subunit of prokaryotic ribosomes. The genes coding for it are referred to as 16s rDNA and are used in reconstructing phylogenies, due to the slow rates of evolution of this region of the gene. Multiple sequences of 16s rRNA can exist within a single bacterium and has a structural role, acting as a scaffold defining the positions of the ribosomal proteins. The 3′ end contains the anti-Shine-Dalgarno sequence, which binds upstream to the AUG start codon on the mRNA. The 3′-end of 16s RNA binds to the proteins S1 and S21, which are involved in initiation of protein synthesis.

The 16s rRNA gene is useful for phylogenetic studies as it is highly conserved between different species of bacteria and archaea.

In addition to highly conserved primer binding sites, 16s rRNA gene sequences contain hypervariable regions that can provide species-specific signature sequences useful for bacterial identification. 16s rRNA gene sequencing is useful for identifying bacteria, and is capable of reclassifying bacteria into completely new species, or even genera, including those that have never been successfully cultured.

Thus, the 16s rRNA gene is used as the standard for classification and identification of microbes, because it is present in most microbes and shows proper changes. Type strains of 16S rRNA gene sequences for most bacteria and archaea are available on public databases, such as NCBI. However, the quality of the sequences found on these databases is often not validated. The sequencing and computer-aided methods of the present invention aim to improve the classification and identification of microbes using 16s rRNA gene sequences. “18s rRNA” refers to 18s ribosomal RNA. 18s rRNA is a component of the small eukaryotic ribosomal subunit (40S). 18s rRNA is the structural RNA for the small component of eukaryotic cytoplasmic ribosomes, and thus one of the basic components of all eukaryotic cells.

18s rRNA is thus effectively the eukaryotic nuclear homologue of 16s ribosomal RNA in prokaryotes and mitochondria.

The genes coding for it are referred to as 18s rDNA and are used in reconstructing the evolutionary history of organisms, especially in vertebrates. The small subunit (SSU) 18s rRNA gene is frequently used gin phylogenetic studies and is useful as a marker for random target polymerase chain reaction (PCR) in environmental biodiversity screening. rRNA gene sequences are easy to access due to highly conserved flanking regions allowing for the use of universal primers. Their repetitive arrangement within the genome provides excessive amounts of template DNA for PCR, even in the smallest organisms. The 18s gene is part of the ribosomal functional core and is exposed to similar selective forces in all living organisms. Therefore, the 18s gene serves as a useful marker for phylogenetic studies. The term “amplification” refers to a mechanism leading to multiple copies of a chromosomal region within a chromosome arm. This includes an increase in the frequency of a gene or chromosomal region, as a result of replicating a DNA segment by an in vivo, ex vivo or in vitro process. Amplification processes envisaged include both artificial amplification processes (occurring ex vivo or in vitro), such as polymerase chain reaction (PCR) and non-artificial amplification processes, such as gene duplication.

PCR is an artificial DNA amplification technique creating multiple copies of small segments of DNA. The term “artificial” is understood to mean that the process does not occur in nature i.e. that human intervention is required, such as by genetic engineering. Thus, the term “artificial amplification” is understood to refer to amplification processes that do not occur in nature, such as PCR.

Non-artificial amplification processes occur in nature, such as gene duplication where a portion of the genetic material is duplicated or replicated resulting in multiple copies of that region. Gene duplication may lead to mutation and certain disorders, and is also an important event in terms of evolution, allowing each gene to evolve independently to possess distinct functions.

“amplification dependent” refers to sample preparation methods using isolated samples requiring a step of amplification, in particular, a step of artificial amplification of the isolated sample, such as by PCR.

The present invention encompasses both methods that are amplification dependent, such as in the PCR-dependent methods of the invention, as well as those methods that are amplification independent (i.e. do not require any amplification step on the isolated sample, such as a PCR-amplification step), such as the RT-SSU rRNA sample preparation methods of the invention. “PCR-independent” refers to methods that do not require a PCR amplification step, such as the RT-SSU rRNA sample preparation methods of the invention.

“PCR amplicon” refers to DNA and/or RNA that is the product of PCT amplification. “RT-SSU rRNA sequencing” refers to direct rRNA sequencing. SSU rRNA are small subunit gene sequences.

In some embodiments of the invention, the SSU rRNA sequencing is amplification dependent. The SSU rRNA is isolated and purified using gel electrophoresis. The SSU rRNA is then reverse transcribed into rDNA or ds cDNA and amplified using reverse transcriptase-PCR (RT-PCR) for subsequent sequencing and classifying using the computer-implemented methods of the present invention.

Such methods of amplifying 16 or 18S rRNA sequences rely on the use of degenerate primers (universal primers) that have been designed to recognise, in a semi-specific manner, all known rRNA sequences. The primers are designed to highly conserved areas of the small subunit ribosomal gene. For example, universal bacteria primers can be used to amplify 16S rRNA by RT-PCR.

In other embodiments of the invention, the SSU rRNA sequencing is amplification independent (artificial or non-artificial amplification). In particular embodiments, the SSU rRNA sequencing is PCR-independent.

The amplification-independent methods of the present invention comprise separating the components in an isolated biological sample according to their size, wherein the components are at least one of DNA and RNA. The method subsequently comprises purifying and isolating the RNA component from the biological sample, followed by isolation and purification of SSU rRNA using a composition comprising a ribonuclease inhibitor and a deoxyribonuclease to remove DNA from the sample. The SSU rRNA is then reverse transcribed into ds cDNA using random primers (this is not an amplification step) for subsequent sequencing and classifying using the computer-implemented methods of the present invention.

“chaotropic agent” refers to a molecule in solution in water that can disrupt the hydrogen bonding network between water molecules. This can denature macromolecules (such as proteins and nucleic acids) by disrupting non-covalent forces such as hydrogen bonds, van der Waals forces and hydrophobic effects. For example, a chaotropic agent can be used to denature DNase and RNase enzymes (and thereby prevent enzymatic DNA and RNA degradation following cell lysis), whilst also allowing for subsequent DNA/RNA binding to silica via salt bridges. Preferably, the chaotropic agent is used at a pH below 7.0.

Chaotropic agents include guanidine thiocyanate, butanol, ethanol, guanidium chloride, lithium perchlorate, lithium acetate, magnesium chloride, phenol, propanol, sodium dodecyl sulphate, thiourea, urea and combinations thereof.

Preferably, the chaotropic agent is a guanidine-based salt. Preferably, the guanidine-based salt is selected from guanidine thiocyanate (GTC) or guanidine hydrochloride or combinations thereof. The preferred guanidine-based salt is guanidine thiocyanate.

“antichaotropic agent” is a molecule in an aqueous solution that will increase the hydrophobic effects within the solution. Antichaotropic salts like ammonium sulphate can be used to precipitate substances from the impure mixture. This is used in protein purification processes, to remove undesired proteins from solution. For example, RNAlater®, utilises the anti-chaotropic agent ammonium sulphate.

“solvent” is a substance that dissolves a solute (a chemically different liquid, solid or gas), resulting in a solution. A solvent is usually a liquid but can also be a solid or a gas. Preferred solvents for use in the contacting composition are selected from isopropanol (IPA or propan-2-ol) or ethanol or combinations thereof.

Additionally, the use of a solvent, solvent selected from isopropanol (IPA/propan-2-ol) or ethanol or combinations thereof, in combination with size-selected silicon dioxide is understood to cause RNA, as well as DNA, to bind strongly to the silica, assisting in providing a dual extraction methodology.

“microbial cell lysis” covers various types of cell lysis including mechanical and chemical lysis. Chemical cell lysis can be performed, for example, using the chaotropic agent itself. Chemical lysis can also be performed using a chaotropic agent and a solvent, such as isopropanol (IPA or propan-2-ol) or ethanol or combinations thereof. The composition may further comprise at least one additional ingredient selected from a buffer, sodium chloride or a detergent (such as Triton X-100) and combinations thereof. Mechanical cell lysis is preferred, such as through the use of a chamber containing beads (such as glass beads) through which the sample is passed. The beads are then spun via a motor means to cause mechanical lysis of the contacted sample. A micro-lyser device can be employed to perform mechanical lysis. Preferably, a battery-powered micro-lyser device is employed.

“size selected silicon dioxide” is understood to mean that the size of the silicon dioxide beads used to bind to DNA/RNA in the sample is selected to optimise the surface area available for DNA/RNA to bind effectively. Preferably, size-selected silicon dioxide beads are used that can be suspended in a solution, such as water.

“ribonuclease inhibitor” (RNase inhibitor) refers to a large (approximately 49 kDa), acidic, leucine-rich repeat protein that forms extremely tight complexes with ribonucleases. It is a major cellular protein, comprising approximately 0.1% of all cellular protein by weight. A wide variety of ribonuclease inhibitors are known to those skilled in the art, such as those RNase inhibitors that inhibit RNAse A, B and C, RNase 1 and T1.

“a deoxyribonuclease” (DNase) refers to any enzyme that catalyzes the hydrolytic cleavage of phosphodiester linkages in the DNA backbone, thus degrading DNA. Deoxyribonucleases are a type of nuclease, a generic term for enzymes capable of hydrolising phosphodiester bonds that link nucleotides. A wide variety of deoxyribonucleases are known to those skilled in the art, such as DNase I and DNase II. “random primer” is used interchangeably with the term “random hexamer”. These are oligonucleotides of six bases with the sequence to prime reverse transcription. This is not part of an amplification step, but serves to prime reverse transcription in the amplification-independent sample preparation methods of the present invention.

Random primers are synthesised entirely randomly to give a large range of sequences that have the potential to anneal at many random points on a DNA or RNA sequence and act as a primer to commence first strand cDNA synthesis.

An “isolated sample” in the context of the sample preparation methods of the present invention is a biological sample that has been isolated from a subject, for example, an isolated tumour sample. The biological sample can include organs, tissues, cells and/or fluids. The isolated sample comprises DNA, RNA or protein or combinations thereof.

The term “subject” refers to any animal, particularly an animal classified as a mammal, including humans, domesticated and farm animals, and zoo, sports, or pet animals, such as dogs, horses, cats, cows, and the like. Preferably, the subject is human.

A “k-mer” is a short DNA/RNA or protein sub-sequence, usually 3 to 8 bases or residues long, but in theory of any size. Any alphabet size and number of different k-mers are accepted.

A “k-mer integer” is a k-mer sub-sequence converted to a unique integer so that all different k-mers have unique integers. This is commonly done in programs because k-mers can then be used as indices in regular arrays.

A “hierarchy” means any multi-level organising skeleton such as hierarchies (with a single parent and multiple children) and ontologies (with multiple parents and multiple children that may not include the parents).

A “group” is a point node in the hierarchy. Groups have parent(s) and children identifiable by unique identifiers (IDs).

A “signature” is a data structure that holds information for a given group, as explained below.

A “signature array” is a list of k-mer id/frequency-of-occurrence pairs. For efficiency they are preferably stored in arrays of [id, frequency, id, frequency, . . . ]. The frequencies have been scaled linearly to a fixed maximum N, i.e. the scaling ratio for all frequencies is N divided by the highest count observed.

A “signature map” is a file based key/value storage where taxon ID is key and stringified signatures are values.

A “sample” in the context of the computer-implemented methods of the invention is a collection of query sequences that are to be classified.

### DETAILED DESCRIPTION OF THE INVENTION

The computer-implemented methods of the invention have the advantage of providing an improved (faster and more accurate) computer-implemented method for sequencing a sample, and handling and analysing large quantities of sequence data in a meaningful way. The computer-implemented methods can usefully handle samples prepared by either the amplification dependent (e.g. PCR amplification) sample preparation methods of the present invention or the amplification-independent (e.g. PCR-independent) sample preparation methods of the present invention.

The sample isolation methods of the invention have the advantage that both DNA and RNA can be isolated at the same time, thus increasing the extraction efficiency.

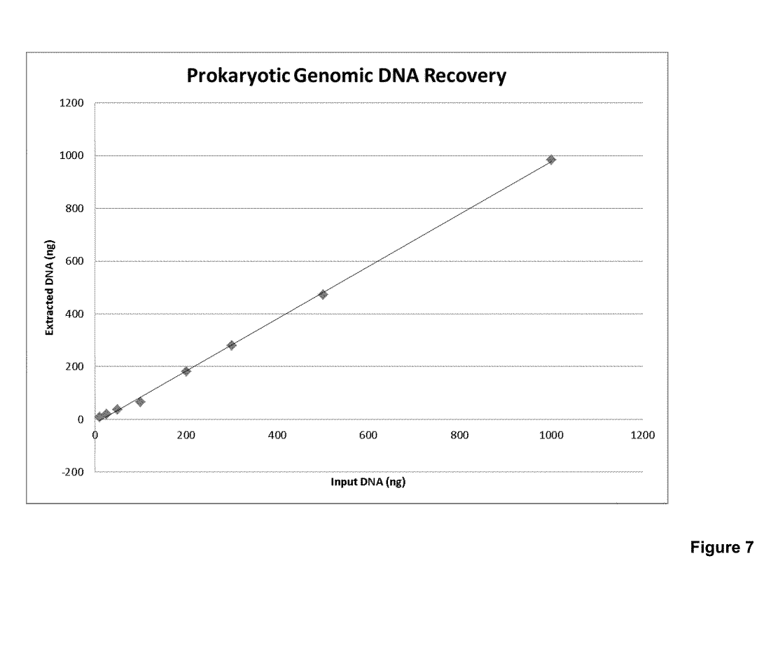

The extraction efficiency of each of DNA and RNA individually is also optimised with the sample isolation methods of the invention. Over the range of the samples typically obtained from the field in the oil and gas industries, the method provides a clear linear relationship between the input levels of biomass (i.e. including DNA/RNA) in samples and the output of purified DNA/RNA (see FIG. 7). This is important if quantitative assays (such as qPCR and RT-qPCR) are to have any validity downstream. In prior art methodologies, this linear relationship does not exist, particularly as the levels of biomass input become very small.

The use of an activated charcoal treatment step during extraction method of the present invention can increase the efficiency of DNA/RNA extraction. The activated charcoal treatment step can be before, simultaneously with or subsequent to contacting the isolated biological sample with a composition comprising a chaotropic agent. The activated charcoal treatment step can be before, simultaneously with or subsequent to subjecting the contacted sample to microbial cell lysis. In some aspects, the activated charcoal treatment step can be before contacting the lysed biological sample with a slurry of size-selected silicon dioxide. Without being bound by theory, it is believed that the use of an activated charcoal treatment step can avoid: (a) the prevention/blocking of DNA/RNA binding to the slurry of size-selected silicon dioxide due to the production chemicals, natural ions and biomolecules etc. present in the isolated biological sample; and/or (b) the co-extraction/concentration of qPCR assay inhibitors in the isolated biological sample, which can prevent qPCR amplification from isolated DNA.

By employing a slurry of size-selected silicon dioxide, large sampling volumes (up to 10 ml) can be processed, which cannot easily, or as effectively, be achieved by traditional silica-based methodologies, such as silica spin filters or membranes. Additionally, the use of size-selected silicon dioxide optimises the surface area available for DNA/RNA to bind effectively, even in large volumes of binding buffer.

The use of mechanical lysis, such as through a micro-lyser device in combination with a chaotropic agent, provides an easy-to-use sampling kit that also provides effective preservation of DNA/RNA in an isolated biological sample in the field, allowing preservation until the sample reaches the destination where it is to be processed, extracted and, ultimately, sequenced. This provides for increased DNA/RNA yield and decreased DNA/RNA fragmentation from samples; in contrast to prior art bulk water and filter-based field sampling methodologies. In particular for samples isolated from gas and oil fields (e.g. oil wells) where the sample processing, extraction and sequencing site is remote from the sampling site.

The sample isolation methods of the invention have the further advantage of being useful with other forms of low copy number and/or damaged and fragmented nucleic acids such as: certain environmental samples; certain kinds of medical samples (e.g. formalin-fixed paraffin-embedded (FFPE) tissues); certain kinds of forensic samples; and certain kinds of ancient DNA and other archaeogenetics samples (i.e. vanishingly small quantities of damaged and fragmented DNA from museum and archaeological samples thousands of years old). The sample isolation methods of the invention are able to produce data from even very low biomass inputs (data not shown) and, moreover, can do so quantitatively.

The use of a chaotropic agent, such as GTC, in combination with a solvent, such as isopropanol (IPA or propan-2-ol), has a number of advantages. Without wishing to be bound by theory, it is understood that the chaotropic agent denatures DNase and RNase enzymes (and thereby prevent enzymatic DNA and RNA degradation following cell lysis), whilst also allowing for subsequent DNA/RNA binding to size-selected silicon dioxide beads via salt bridges, preferably at a pH of less than 7.0. It is further understood that the solvent causes RNA, as well as DNA, to bind strongly, and quantitatively, to the silicon dioxide, thus making it a dual extraction methodology.

The amplification-independent sample preparation methods of the present invention have the further advantage that inherent biases are significantly reduced, providing a higher quality sample. The PCR-independent sample preparation methods of the present invention can optionally be used in conjunction with existing sequencing methods or the computer-implemented methods of the present invention. In one embodiment of the invention, the amplification-independent sample preparation methods of the present invention can be used in conjunction with the computer-implemented methods of the present invention to advantageously provide faster and more accurate sequencing classification with significant reduction of inherent biases.

The novel methods of the invention can, in particular, be used to address the challenges of assessing biological diversity. In one aspect, the present invention provides a method that provides a specific, unbiased and global assessment of the SSU rRNA diversity and relative abundance within microbial communities across Bacteria, Archaea and Eukarya, simultaneously from the same sample.

The present invention has particular utility in the oil and gas fields, in particular, in classifying microbial diversity in biological samples isolated from oil wells.

Using the canine oral microbiome as the test bed alongside a novel computer-implemented method, the inventors were able to determine a heretofore-unseen level of diversity and population structure from sequences obtained directly from ribosomal RNA generated without any in vitro amplification steps. The present invention provides a platform for a new era in molecular microbial ecology in which the artificial amplification of taxonomic marker sequences is neither necessary nor desirable.

The present inventors sequenced a library composed entirely of reverse-transcribed SSU rRNA (RT-SSU rRNA) molecules from the canine oral microbiome, and compared the sequence composition with a PCR amplicon library generated from the same sample using the novel taxonomic classification computer-implemented methods of the present invention. The present inventors found that the direct RT-SSU rRNA sequencing and computer-implemented methods of the present invention detected greater taxonomic diversity, provided comparative rRNA abundance data across all three domains of life, and detected taxa not recognised by ‘universal’ primer sets.

1. Sample Isolation and Preparation Methods of the Invention

The sample isolation and preparation methods of the invention encompass both amplification-dependent methods and amplification-independent methods. These methods can usefully be combined with the computer-implemented methods of the present invention.

1.1 Sample Isolation Methods

In the present invention, there is provided a method for processing an isolated biological sample containing at least one of DNA and RNA, such that the DNA and/or RNA is preserved in the sample at ambient temperatures for at least thirty days, the method comprising: contacting the isolated biological sample with a composition comprising a chaotropic agent, and subjecting the contacted sample to microbial cell lysis.

DNA/RNA survives in the lysed sample at ambient temperatures for at least one day to at least one week. More preferably, the DNA and/or RNA can be preserved in the sample at ambient temperatures for at least one week, two weeks, three weeks or four weeks. More preferably, the DNA and/or RNA can be preserved in the sample at ambient temperatures for at least one month.

The method can include the further step of subjecting the isolated biological sample to an activated charcoal treatment.

The isolated biological sample can be subjected to the activated charcoal treatment step before, simultaneously with or subsequent to contacting the sample with the chaotropic agent. For example, the activated charcoal treatment could be performed immediately before the sample is contacted with the chaotropic agent or at the same time as the chaotropic agent is contacted with the sample. The activated charcoal treatment could be performed after the sample has been contacted with the chaotropic agent and before the sample is subjected to microbial cell lysis.

The isolated biological sample can be subjected to the activated charcoal treatment step before, simultaneously with or subsequent to subjecting the contacted sample to microbial cell lysis. For example, the activated charcoal treatment could be performed immediately before microbial cell lysis or at the same time as microbial cells lysis. The activated charcoal treatment could be performed after microbial cell lysis. In some aspects, the isolated biological sample is subjected to the activated charcoal treatment before contacting the lysed biological sample with a slurry of size-selected silicon dioxide. For example, the activated charcoal treatment could be performed after microbial cell lysis and before contacting the lysed biological sample with a slurry of size-selected silicon dioxide.

The activated charcoal treatment could be used multiple times during the sample isolation and extraction methods of the present invention.

According to another aspect of the present invention, there is provided a method for preparing an isolated biological sample containing at least one of DNA and RNA, the method comprising:
(i) contacting the isolated biological sample with a composition comprising a chaotropic agent, and subjecting the contacted sample to microbial cell lysis;
(ii) contacting the lysed biological sample with a slurry of size-selected silicon dioxide to form at least one of DNA-silicon dioxide complexes or RNA-silicon dioxide complexes (or both) in the sample;
(iii) isolating at least one of DNA-silicon dioxide complexes or RNA-silicon dioxide complexes from the sample; and
(iv) separating at least one of DNA and RNA from the silicon dioxide and collecting at least one of the DNA and RNA.

Once processed, fully collected and extracted DNA/RNA survives for at least months at −80° C., preferably, at least one month.

This method can also include the further step of subjecting the isolated biological sample to an activated charcoal treatment.

The isolated biological sample can be subjected to the activated charcoal treatment step before, simultaneously with or subsequent to contacting the sample with the chaotropic agent. For example, the activated charcoal treatment could be performed immediately before the sample is contacted with the chaotropic agent or at the same time as the chaotropic agent is contacted with the sample. The activated charcoal treatment could be performed after the sample has been contacted with the chaotropic agent and before the sample is subjected to microbial cell lysis.

The isolated biological sample can be subjected to the activated charcoal treatment step before, simultaneously with or subsequent to subjecting the contacted sample to microbial cell lysis. For example, the activated charcoal treatment could be performed immediately before microbial cell lysis or at the same time as microbial cells lysis. The activated charcoal treatment could be performed after microbial cell lysis. In some aspects, the isolated biological sample is subjected to the activated charcoal treatment before contacting the lysed biological sample with a slurry of size-selected silicon dioxide. For example, the activated charcoal treatment could be performed after microbial cell lysis and before contacting the lysed biological sample with a slurry of size-selected silicon dioxide.

The activated charcoal treatment could be used multiple times during the sample isolation and extraction methods of the present invention. These sample isolation methods can be used in combination with the amplification-dependent sample preparation methods and amplification-independent sample preparation methods of the invention. These sample isolation methods can be used upstream of the amplification-dependent methods and amplification-independent methods of the invention to provide higher quality samples for the amplification-dependent methods and amplification-independent methods due to decreased fragmentation and degradation of at least one of DNA and RNA in the isolated biological sample. These methods have particular utility in combination with the amplification-independent sample preparation methods.

In combination, such methods advantageously provide a fast and accurate method for sample preparation and allow the classification of sequences in samples into a large collection of classified homologous sequences.

In particular, it is possible to detect novel centres of variation within Bacteria, Archaea and Eukarya, and these data could be used to design and optimise more inclusive taxon-specific PCR, qPCR and RT-qPCR primer sets and probes for a more detailed investigation of their taxonomy and ecology.

The present invention has particular utility in the oil and gas industries, in particular, in classifying the microbial diversity in biological samples isolated from oil wells.

The sample isolation methods of the present invention are also useful in other downstream processes in molecular biology, such as RT-qPCR and qPCR. Exemplary uses include use in RT-qPCR and qPCR for key gene targets related to biocorrosion.

FIG. 7 shows input-versus-output over orders-of-magnitude differences in prokaryotic genomic DNA recovery from an oil and gas industry low biomass environmental sample—using the sample isolation methods of the present invention.

In one embodiment, guanidine thiocyanate (GTC) is used as the chaotropic agent in the transportation buffer. GTC has particularly strong RNase inhibitory properties and also acts as an effective binding buffer, particularly when supplemented with various additional reagents. An alternative chaoptropic agent is guanidine hydrochloride.

Additional components can be added to the composition comprising a chaotropic agent, which is used as the transportation buffer.

An exemplary additional component is a solvent, such as and isopropanol (IPA or propan-2-ol) or ethanol. The combination of GTC and IPA is envisaged.

Exemplary additional components are shown in the table below:

| Additional Components | Volume | Final Concentrations |
|---|---|---|
| 0.5M Tris Buffer pH 6.8 | 20.6 ml | 18.35 mM |
| 5M NaCl | 2.84 ml | 25.3 mM |
| Triton X-100 | 7.39 ml | 1.32% |
| Isopropanol/IPA/Propan-2-ol | 30.4 ml | 5.42% |

The chaoptropic agents and mechanical lysis of the sample isolation methods of the present invention can conveniently be employed in a kit which can be used in the field, in industries such as the oil and gas industries. The collected, processed and preserved sample can then be transported to a laboratory for downstream sample processing, extraction and sequencing methods.

GTC is preferably shipped in light-protective containers with PTFE-lined polypropylene screw caps (to prevent leakage).

Preferably, about 10 ml of biological sample (for example planktonic microbial cells in produced water), is added to 40 ml of the composition comprising GTC to give a final 50 ml volume.

The sample can optionally undergo an activated charcoal treatment.

By way of example, the sample could undergo an activated charcoal treatment before, simultaneously with or subsequent to contacting the sample with the chaotropic agent.

For example, the activated charcoal treatment could be performed immediately before the sample is contacted with the chaotropic agent or at the same time as the chaotropic agent is contacted with the sample. The activated charcoal treatment could be performed after the sample has been contacted with the chaotropic agent and before the sample is subjected to microbial cell lysis.

By way of a further example, the sample could undergo an activated charcoal treatment before, simultaneously with or subsequent to subjecting the contacted sample to microbial cell lysis. For example, the activated charcoal treatment could be performed immediately before microbial cell lysis or at the same time as microbial cells lysis. The activated charcoal treatment could be performed after microbial cell lysis. In some aspects, the isolated biological sample is subjected to the activated charcoal treatment before contacting the lysed biological sample with a slurry of size-selected silicon dioxide. For example, the activated charcoal treatment could be performed after microbial cell lysis and before contacting the lysed biological sample with a slurry of size-selected silicon dioxide.

The activated charcoal treatment could be used multiple times during the sample isolation and extraction methods of the present invention.

The sample is then mixed by inversion and subjected to mechanical lysis. Mechanical lysis can be performed by a micro-lyser device. In one such device, a motor spins small glass beads around a chamber that the sample passes through. The glass beads perform the mechanical lysis in a continuous fashion as the full 50 ml sample is passed through. The sample is passed twice (in both directions) through a micro-lyser device to effect microbial lysis and the simultaneous inactivation of intracellular and extracellular DNase and RNase enzymes. Preferably, a micro-lyser device is used, which has means to connect the device to a container (such as a syringe) which can process the biological sample.

The DNA and RNA in the lysed sample is preserved long-term (for at least one week and preferably, up to thirty days) at ambient temperatures.

The table below provides an exemplary composition comprising GTC for microbial cell lysis and sample preservation:

| | | Final |
|---|---|---|
| Component | Volume (per sample) | Concentration* |
| 6M Guanidine Thiocyanate | 33.333 ml | 5M |
| Millipore-Quality dH₂O | 1.937 ml | — |
| 0.5M Tris Buffer pH 6.8 | 1.450 ml | 18.1 mM |
| 5M NaCl | 0.200 ml | 25.0 mM |
| Triton X-100 | 0.520 ml | 1.3% |
| Isopropanol/IPA/Propan-2-ol | 2.140 ml | 5.4% |

Once the sample has been preserved in the field, it can be transported to a laboratory for further sample processing and DNA/RNA isolation. This transportation typically could take weeks (or even months) before the sample is further processed. The present invention provides stable samples where the DNA/RNA integrity and yield is preserved during this period.

In the present invention, further processing of the stabilised sample can occur using size-selected silicon dioxide beads.

First, a solvent, such as isopropanol (IPA or propan-2-ol), can be added to the GTC-contacted sample. The addition of a further solvent to the silica binding step increases the efficiency of RNA binding (in particular) to the silica. Without the additional solvent, mostly DNA (with much reduced amounts of RNA) are typically recovered.

Preferably, the pH of the mixture is less than 7.0. Optionally, a pH indicator, such as Cresol Red Indicator Buffer, can be added to the mixture. If the pH is higher than 7.0 (via the indicator colour change), the pH can be adjusted downwards, for example with sodium acetate or acetic acid, until the indicator shows pH<7.0.

The sample can then be contacted with size-selected silicon dioxide. The contacted sample can then be rotated overnight at room temperature to allow DNA/RNA binding to silica to take place.

The silicon dioxide beads that are complexed with DNA/RNA are pelleted by centrifugation. Repeated washing steps, for example with 70-80% ethanol, can be performed and, finally, the beads resuspended in an appropriate aqueous buffer and the extracted DNA/RNA recovered.

The DNA/RNA can be eluted from the beads using an aqueous resuspension buffer (such as Qiagen's Buffer EB/RNasin Resuspension Buffer) at about 50° C. The sample is further centrifuged and the liquid phase recovered, which contains the isolated DNA/RNA for downstream processing, such as NGS library preparation, PCR, qPCR and RT-qPCR.

1.2 Amplification-Dependent Sample Preparation Methods

The amplification-dependent sample preparation methods of the present invention, such as PCR-dependent sample preparation methods, comprise separating the components in an isolated biological sample according to their size, wherein the components are at least one of DNA and RNA. The method subsequently comprises purifying and isolating the RNA component from the biological sample, followed by isolation and purification of SSU rRNA using gel electrophoresis. The SSU rRNA is then reverse transcribed into rDNA or ds cDNA and amplified using reverse transcriptase-PCR (RT-PCR) for subsequent sequencing in the computer-implemented methods of the present invention.

Such methods of amplifying 16 or 18S rRNA sequences rely on the use of degenerate primers (universal primers) that have been designed to recognise, in a semi-specific manner, all known rRNA sequences. The primers are designed to highly conserved areas of the small subunit ribosomal gene. For example, universal bacteria primers can be used to amplify 16S rRNA by RT-PCR.

Such methods of amplifying 16 or 18S rRNA sequences rely on the use of degenerate primers (universal primers) that have been designed to recognise, in a semi-specific manner, all known rRNA sequences. The primers are designed to highly conserved areas of the small subunit ribosomal gene. For example, universal bacteria primers can be used to amplify 16S rRNA by RT-PCR.

The sample produced by the amplification-dependent methods of the present invention can be sequenced using existing sequencing methods and classified using existing methods or, advantageously, the computer-implemented methods of the present invention to obtain classification information to determine microbial diversity.

The amplification-dependent sample preparation methods of the present invention are useful in preparing samples for sequencing and classifying using the computer-implemented methods of the present invention. Such methods advantageously allow the classification of sequences in samples into a large collection of classified homologous sequences.

In particular, it is possible to detect novel centres of variation within Bacteria, Archaea and Eukarya, and these data could be used to design and optimise more inclusive taxon-specific PCR primer sets and probes for a more detailed investigation of their taxonomy and ecology.

The present invention has particular utility in the oil and gas fields, in particular, in classifying microbial diversity in biological samples isolated from oil wells.

1.3 Amplification-Independent Sample Preparation Methods

While the above amplification-dependent methods of the invention are useful in preparing samples for sequencing and classifying using the computer-implemented methods of the present invention, such methods involving the amplification of 16 or 18S rRNA sequences rely on the use of degenerate primers (universal primers) that have been designed to recognise, in a semi-specific manner, all known rRNA sequences. They are designed to highly conserved areas of the small subunit ribosomal gene. However, it has previously been found that universal primers are not truly universal and as much as half of the microbial diversity is likely to be missed by currently designed primers. Thus, “true” universals primers cannot be generated.

The amplification-independent sample preparation methods of the present invention aim to avoid such loss of diversity.

In one embodiment of the amplification-independent (e.g. PCR-independent) methods of the present invention, the method is used to characterise SSU rRNA genes derived from all members of the microbial community. The method can be used to sequence a library composed entirely of SSU rRNA molecules, without an amplification step (e.g. a universal PCR amplification step), to provide much-extended catalogue of microbial diversity with differing population structure.

In one embodiment, the amplification-independent methods of the present invention comprise separating the components in an isolated biological sample according to their size, wherein the components are at least one of DNA and RNA. The method subsequently comprises purifying and isolating the RNA component from the biological sample, followed by isolation and purification of Small Sub-Unit ribosomal RNA (SSU rRNA) using a composition comprising a ribonuclease inhibitor and a deoxyribonuclease to remove DNA from the sample. The SSU rRNA is then reverse transcribed into ds cDNA using random primers. This is not an amplification step. Multiple copies are not generated during this reverse transcription step. The random primers serve only to prime the reverse transcription step.

Advantageously, the use of random primers serves to reduce the loss in diversity and inherent bias.

In embodiments of the method, total RNA can be isolated from an isolated biological sample followed by the isolation of total RNA from the SSU rRNA (SSU 16S or 18S rRNA). Random primers for SSU rRNA can then be used as the base for reverse transcription of the SSU rRNA.

The sample produced by the amplification-independent methods of the present invention can then be sequenced using existing sequencing methods and classified using existing classification methods or, advantageously, the computer-implemented methods of the present invention to obtain classification information to determine microbial diversity.

The PCR-independent methods of the present invention do not use any amplification step (e.g. an artificial amplification step), such as PCR amplification.

The amplification-independent sample preparation methods of the present invention are useful in preparing samples for sequencing and classifying using the computer-implemented methods of the present invention. Such methods advantageously provide a fast and accurate method for sample preparation and allow the classification of sequences in samples into a large collection of classified homologous sequences.

In particular, it is possible to detect novel centres of variation within Bacteria, Archaea and Eukarya, and these data could be used to design and optimise more inclusive taxon-specific PCR primer sets and probes for a more detailed investigation of their taxonomy and ecology.

The present invention has particular utility in the oil and gas fields, in particular, in classifying microbial diversity in biological samples isolated from oil wells.

More specifically, SSU rRNA molecules can be fractionated using agarose gel electrophoresis, reverse-transcribed and converted to double-stranded cDNA that is fragmented for library generation and directly sequenced using the computer-implemented methods of the present invention.

In embodiments of the method, following isolation of a biological sample, desired components (e.g. DNA, RNA or protein or combinations thereof) can be extracted from the sample. The components can be separated, for example, by size separation using existing methods. In one embodiment, gel electrophoresis is used for size separation. Genomic DNA (greater than or equal to 20 Kb) and/or SSU rRNA (about 1 Kb) can be excised from the gel and then purified. DNA can be purified using existing methods in the art. In one embodiment, SSU rRNA is purified using a ribonuclease inhibitor and a deoxyribonuclease, such as Turbo DNA0free (Ambion). The SSU rRNA is precipitated, centrifuged and resuspended.

Following the purifying step, SSU rRNA can be reverse transcribed using random primers to produce the corresponding ds cDNA to prepare the sample, which can subsequently be used for sequencing.

In certain embodiments, SSU rRNA is separated by electrophoresis of SSU rRNA and large subunit (23S/28S) rRNA bands.

Random primers are used to reverse transcribe SSU rRNA to produce double stranded (ds) cDNA.

In preferred embodiments, no amplification occurs when the SSU rRNA is reverse transcribed using random primers to produce ds cDNA.

In certain embodiments, the produced ds cDNA can be artificially amplified, such as by existing PCR amplification methods, to prepare the sample, which can subsequently be used for sequencing.

In preferred embodiments, no amplification step (artificial or non-artificial) is employed in the sample preparation method. In other words, no amplification occurs at any point in the sample preparation method.

The amplification-independent methods of the present invention have several advantages, in particular, they provide for the fast and accurate analysis of microbial diversity in isolated biological samples. The methods are simple and low cost. The methods do not require amplification (e.g. PCR amplification), reducing the inherent bias. Since no amplification step is required, the method can be used for accurate quantification during the classification steps. For example, the ratio of various microbial species can be accurately quantified.

This methodology takes advantage of the fact that ribosomal RNA is very abundant in the cell. Although variations in the number of rRNA gene copies in the genome and the number of SSU rRNA molecules transcribed (a proxy for metabolic activity) for each species being studied will undoubtedly affect the read density of species detected, it is believed that direct RT-SSU rRNA sequencing has merit for inferring relative species abundance in situ. This is because, unlike DNA-based PCR approaches, this technique will specifically detect the rRNA molecules of species within the microbiome. Direct sequencing of rRNA molecules has the advantage of avoiding PCR-associated biases, primer mismatches and is more likely to identify ‘active’ species of importance within the microbiome that can be further validated by complimentary approaches.

In one aspect, methods of the invention can be applied to SSU rRNA extracted from canine plaque samples.

The microbial diversity and abundances resulting from the amplification-independent methods of the invention can be compared to those obtained from a PCR amplicon-derived library (an amplification-dependent method of the invention). The amplicon library was prepared using a universal bacterial primer pair targeting an approximately 460 bp region of the 16S rRNA gene containing the variable regions 1-3, and the DNA serving as the template was extracted simultaneously with RNA from the same plaque sample. Several sets of universal bacterial 16S rRNA gene PCR primers that are commercially available can be employed. This primer pair was selected because it has specificity for all cloned sequences within a general bacterial 16S rRNA gene clone library derived from the canine oral cavity, and in silico comparative taxonomic classification of these cloned sequences corresponding to V1-3, V5-V6 and V4 regions demonstrated that the V1-3 amplicon provided the greatest taxonomic resolution of the samples and the longest amplicon length compared to the other ‘universal’ primer sets.

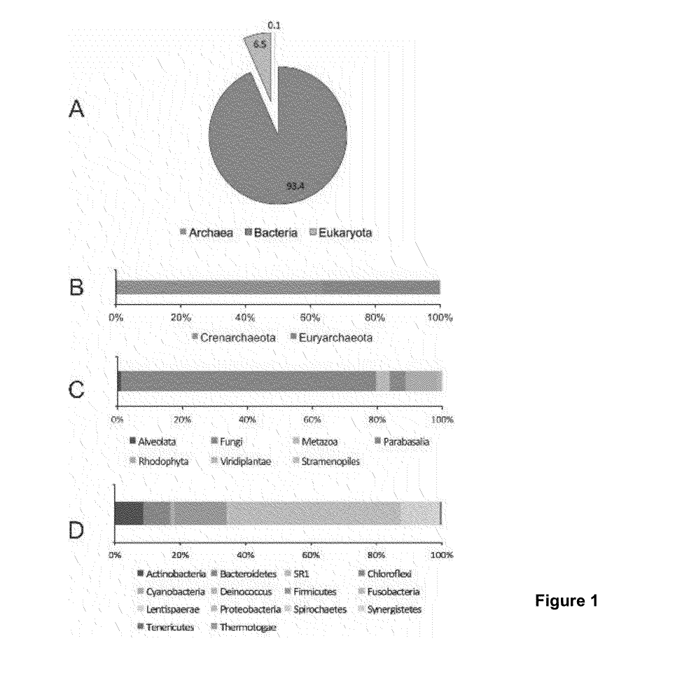

SSU rRNA relative abundances determined by the amplification-independent RT-SSU rRNA sequencing approach of the present invention revealed a canine oral microbiota dominated by Bacteria (93.4%) with only a small proportion of archaeal (0.1%) and eukaryotic (6.5%) SSU rRNA detected (FIG. 1A). This is consistent with previous findings that Archaea represent only a very small fraction of the oral microbiome with diversity restricted to a few phylotypes. However, whereas previous studies suggest that all oral Archaea are methanogenic members of the phylum Euryarchaeota, the amplification-independent RT-SSU rRNA approach of the present invention detected a greater abundance of crenarchaeotes than euryarchaeotes (FIG. 1B); the former are a major archaeal phylum (Crenarchaeota) whose presence in the oral cavity has not previously been reported.

Crenarchaeotes have been detected in human faecal samples using 16S rRNA gene targeted PCR, but attempts to detect this phylum in the oral microbiome using the same approach were unsuccessful. Therefore, the PCR-independent methods of the present invention provide a particularly sensitive method for isolating and preparing biological samples used for detecting biological diversity.

Eukarya represented 6.5% of the total SSU rRNA in the canine plaque samples, and these sequences represented members of several phyla of fungi and protozoa that have been previously detected in the oral cavity (FIG. 1C). The eukaryotic population was dominated by fungi of the subkingdom Dikarya (relative eukaryotic-specific abundance ˜79%), which contains several genera of yeasts that are well established as members of the oral microbiome. Various members of the Metazoa were identified, with Chordata (which contains the genus, Canis) sequences representing 7.75% of the total Metazoan sequences obtained (0.33% of the total Eukaryotic sequences). Taxa containing protozoan species (Aloveolata & Parabasalia), such as Trichomonas (Parabasalia), were also abundant (5%) and surprisingly, sequences related to phyla containing red and green algae/plants were also detected (Rhodophyta, 10%, and Viridiplantae, 1%).

A search of bacterial SSU rRNA sequences against the SILVA database revealed that members of the bacterial phyla Actinobacteria, Bacteroidetes, Firmicutes, Proteobacteria, Spirochaetes, Synergistetes and Tenericutes were the most abundant (˜97% of total bacterial SSU rRNA) (FIG. 1D).

The data in FIG. 1 therefore confirms that pyrosequencing cDNA generated by reverse transcription of fractionated 16S and 18S rRNA according to the methods of the present invention can simultaneously resolve the identity and relative abundance of major microbial taxa across all three domains of life in a single sample.

Furthermore, the amplification-independent RT-SSU rRNA sequencing approach of the present invention detected novel centres of variation within Bacteria, Archaea and Eukarya, and these data could be used to design and optimise more inclusive taxon-specific PCR primer sets and probes for a more detailed investigation of their taxonomy and ecology.

2. Computer Implemented Methods of the Present Invention

The samples prepared by the sample preparation methods of the present invention are sequenced using known sequencing methods. The sequences are then classified using the computer implemented methods of the present invention.

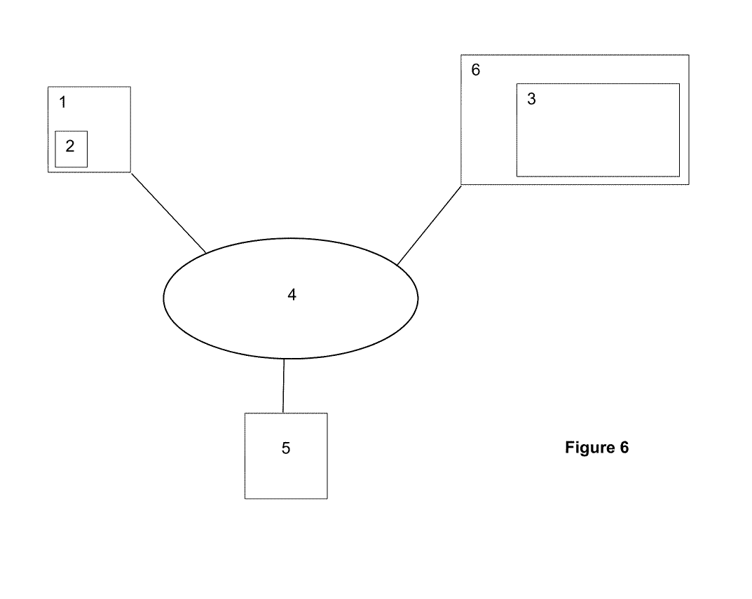

Referring to FIG. 6 of the accompanying drawings, an embodiment of the invention includes a computing device 1. The computing device 1 is configured to perform one or more functions or processes based on instructions which are received by a processor 2 of the computing device 1. The one or more instructions may be stored on a tangible computer readable medium 3 which is part of the computing device 1. In some embodiments, the tangible computer readable medium 3 is remote from the computing device 1 but may be communicatively coupled therewith via a network 4 (such as the Internet).

In embodiments, the computing device 1 is configured to perform one or more functions or processes on a dataset. The dataset may be stored on the tangible computer readable medium 3 or may be stored on a separate tangible computer readable medium 5 (which, like the tangible computer readable medium 3, may be part of or remote from the computing device 1). The tangible computer readable medium 5 and/or the tangible computer readable medium 3 may comprise an array of storage media. In some embodiments, the tangible computer readable medium 3 and/or the separate tangible computer readable medium 5 may be provided as part of a server 6 which is remote from the computing device 1 (but coupled thereto over the network 4).

The one or more instructions, in embodiments of the invention, cause the computing device 1 to perform operations in relation to 16S rRNA or other RNA, DNA or protein reference datasets.

In particular, the one or more instructions may cause the computing device 1 to analyse a 16S rRNA dataset to classify sequences captured by the dataset. The analysis is described below.

2.1 Sequence Classification

The analysis performed by an embodiment of the invention seeks to improve the speed and accuracy of the classification of a new sequence into a large collection of classified homologous sequences, with support for non-amplicon data. An example application is microbial diagnostics and microbiome analyses common in ecology and medicine, where quick and accurate speciation is desired. Another example is functional classification, where there is an ontology of functions rather than a taxonomic hierarchy. The methods of embodiments of the invention cover “signature maps” and preferably also “cluster mapping” which are described in detail below. Prototypes have been implemented in practice and improved results confirmed with 16S rRNA reference databases.

The computer-implemented methods of the present invention create taxonomic overviews from raw sequence data. The computer-implemented methods of the present invention clean and de-replicate sequence reads, detect chimeras in PCR amplicon datasets, calculates similarities and projects these onto the taxonomy of a reference database. The computer-implemented methods of the present invention handle low quality sequences well (ignores low quality regions without discarding the entire sequence read), can detect sequences with low similarity scores, can often differentiate species, works with non-amplicon data, installs from sources with a single line and is fast.

The computer-implemented methods of the present invention are particularly useful for rRNA based bacterial community analysis. The computer-implemented methods of the present invention process, quality check and classify RT-SSU rRNA sequence reads generated using the amplification-dependent (e.g. PCR-dependent) and amplification-independent methods of the present invention, to address the bioinformatics challenge presented by the heterogeneous nature of fragmented RT-SSU rRNA libraries.

2.2 Signature Maps

### 2.2.1 Map Construction

Inputs. Any number of un-aligned sequences, each with hierarchy groups attached, such as those from the National Centre for Biotechnology Information (NCBI) or other reference databases. The sequences may be from the same single molecule and partial sequences can originate from any random region of that molecule. If multiple reference molecules are used, then multiple signature maps should be made.
Sequence access. Sequences are indexed with their IDs as key, so random sets of sequences can be loaded into memory quickly by their IDs.
Signature structure. A signature data structure preferably has one or more of these fields: a group or sequence ID used as retrieval key, a free-format title, ID of the parent group, a list of children IDs, a group signature array and a non-group signature array. The group array holds the k-mers with the most increased frequencies in a given group relative to its “siblings”. Conversely, the non-group array holds k-mers with the most decreased frequencies in a group relative to its siblings.
Hierarchy skeleton. The signature map is initialised with an organising skeleton. Sequences are preferably read in small batches, such as 1,000 at a time, and a hierarchy is generated in memory that exactly spans the groups that come with the batch sequences. Those hierarchy nodes are then preferably stringified and saved in storage for each batch of sequences. The result is a key/value storage map where each entry can be loaded quickly into memory by its unique group ID.
Hierarchy extension. Due to incomplete curation and other reasons, a reference database sometimes has thousands of sequences placed under a single group, perhaps named “unclassified”. The high diversity of such sequences makes it difficult to create signatures for that group. One solution is to form sub-groups within the signature map: whenever there are three or more sequences under a given group, these sequences are clustered into one or more sub-groups, each with its own taxonomy ID and signature structure as above. These sub-groups are often a necessary extension of the skeleton that reference databases provide.
Signature arrays. Group- and non-group signature arrays are preferably filled with frequencies by navigating the whole taxonomic skeleton “depth first” in a recursive fashion: first the top node is loaded into memory, then the first of its children, and so on, until a node is encountered that have no child groups. While at a given group the processing happens, as outlined below. When done the navigation returns to the level above, and so on, until all groups have been visited. In one embodiment, the processing for a given group node (the parent) and its children comprises of these steps:
1) Signature arrays for all children are added, scaled to a fixed maximum N and is attached to the parent node. The result array has the same format as the child signature arrays. If there are sequences among the children, then these are loaded from their indexed storage and converted to the same signature array format before being added. Call this children sum array “children sums” and the equivalent array for each child for “child sum”.
2) The signature group array is then generated for each child in these steps:
a) A “siblings sum” array is derived from the children sums by subtracting the “child-sum” from the children sums.
b) The group array is filled with the child k-mer/frequency pairs with the highest increase in frequency over their siblings. In one embodiment, this is done by “binning”, a common practice in programs. The frequencies with the highest increase are selected, up to a user-given number or percentage, whichever is greater.
c) The group array is scaled to the same fixed maximum value N, as above.
3) The signature non-group array is generated the same way as the group array, except the kept frequencies are those that increase in the siblings sum.
   Performance. Building a map from one million 16S rRNA sequences of 500-1000 bases in length from the RDP project takes 10-20 minutes on commodity hardware. Processing time very much depends on number and sizes of unclassified groups. RAM usage is usually less than one gigabyte and does not depend much on the number of input sequences, as only small sequence batches are loaded at a time. The file size of the resulting signature map is from 1-2 times the sequence file size, depending on user settings.

### 2.2.2 Map Search

The signature map can be searched in a number of ways, with different scoring schemes and logic.

Basic logic. To classify a given query sequence, first compare against the top-level child signatures, then against the best matching child or children, and so on until no signature(s) match much better than others. In essence the signature map is used a “road-map” where higher level signatures tell which turns to take and which groups to skip.
Match score. The similarity between a sequence and a signature is calculated by first finding the set of k-mers shared with the signature group-array (call that set X) and the set not shared with the siblings array (Y). For X and Y the total sum of frequencies (call it S) are calculated. S is finally divided by the set sizes of X and Y. This yields a number between 0 and 1. Alternatively the number of k-mers in the query sequence can be used for division, and yet other scores are possible.
Settings. The user can control minimum output similarity, highest output similarities range, highest similarities range for alternatives, number of levels to try for alternatives, maximum alternatives to try per level and maximum number of output similarities. An embodiment of the invention is preferably operable to ignore (controlled by user settings) low quality spots in both query and reference sequences.

2.3 Cluster Mapping

### 2.3.1 Current Approaches

Sample sequences are commonly analysed in two different ways. One way is clustering of the sequences within each sample producing a set of OTU-clusters for each sample, then mapping these clusters across samples. The variation among all OTUs (known or unknown) can be seen. No reference database is involved. The second way is mapping either OTU cluster representatives or all sample sequences against a reference database.

### 2.3.2 Single Method

To more properly handle the sequences that are more similar to themselves than to anything in the reference database an embodiment of the invention merges multiple steps into one step. The steps preferably comprise:
a) Cluster all sequences amongst themselves, within each sample, requiring e.g. 97% minimum sequence similarity within clusters.
b) Map typical cluster representatives (call them “centre” sequences) to the reference hierarchy using either plain similarities or the signature map described here.
c) Extend the reference hierarchy and sequences with these centre sequences. This can be done “on-the-fly” for the ongoing analysis only, or it can be done permanently as a growing local database to be used by analyses of future samples.
d) Map the remaining non-centre query sequences to the union of the reference database and the centre-sequences. If a given query is most similar to the centre-sequences it will settle in those groups, but if there are higher similarities (or better signatures) elsewhere in the reference data, then it will settle there instead.

The advantages are that all query sequences are optimally placed and users get a single overview. This combined approach greatly reduces the number of low-scoring groups and in combination with the signature map it creates a much clearer picture.

2.4 Map Advantages

### 2.4.1 Speed

a) Searching all query sequences against all reference sequences is a heavy computation and with the volumes of data produced it will become much heavier. It does simply not scale and prevents smaller devices from being able to perform analyses locally as they should. In a signature map search on the other hand, only a small fraction of the reference data are being searched. The search speed also does not depend very much on the size of the reference data.
b) Typically only 20-100 signature k-mers need be checked against the query sequence as opposed to 500 or 1000 or more if the whole reference sequence was used. This reduces the number of comparisons by five times on the average perhaps.

### 2.4.2 Memory

a) Classifying a single sequence at a time requires just a tiny amount of memory, in theory. But in practice, it is faster to keep the parts of the signature map in memory with which there have been matches previously. While in theory this could lead to high memory usage the sample sequences usually fall into a few groups (hundreds or thousands at most) rather than being from every group in the reference database. However a proper application should manage the cache and be able to remove the signatures that have been least frequently matched against.

### 2.4.3 Accuracy

a) Consider two 1000 base long sequences A and B that are identical except for one mismatch near the start. Their sequence similarity would be 99.9%. If the mismatch was at the ends or anywhere else, the similarity as returned by blast and all other programs would still be 99.9% even though all different k-mers are involved. But since the signature map records k-mer/frequency pairs, similarity is highly position dependent as it should be. In practice it means that sequences can be separated by just a single difference. Whether that difference is reliable and informative is another question.
b) One embodiment of the invention is operable to ignore low-quality portions of the query sequence and this does matter in practice.

### 2.4.4 Robustness

a) Group k-mers with low frequency typically do not make it to the parent levels, i.e. group signatures are more conserved than the group sequences as a whole. For this reason query sequence with low overall reference similarity will often succeed where similarity scanning fails. There will be fewer false matches than if the query was compared against all reference data with its higher rate of chance matches.
b) Sequences placed incorrectly in the reference hierarchy can confuse similarity approaches since the highest score may return the wrong group(s), causing wrong classification or loss of accuracy. The signature map eliminates that possibility since incorrectly places sequences are relatively rare so that their k-mers have only a low frequency.

### 2.4.5 Flexibility

a) Non-amplicon query data. Currently most single-gene sequencing is done by amplifying a select part of the gene in order to get enough DNA for the sequencing device. But sequencing hardware and laboratory techniques are emerging that require smaller amounts and cover the whole gene with random reads. The signature map supports this provided there are conserved group specific k-mers in several different positions along the reference molecule. There usually are, but some of the random reads may of course fall into a region where there are no discriminatory reference k-mers, so the classification accuracy (the “resolution”) will be quite different between reads. However, as long as the reads are truly randomly distributed we can simply discard the reads that do not classify to the desired level.
b) Reference data. Partial sequences are often placed in the same group. Sometimes they are from the same molecule region, sometimes not—a difficult problem. The current classifiers have statistical bias towards groups with many sequences and do not handle the situation well. The clustering done as part of the signature map construction merges identical and very similar sequences. The new groups created have k-mer frequencies that are not biased towards groups with many sequences. This remedies part of the problem, but not all. However in the coming years partial sequences will likely be replaced with full-length ones, so the problem should slowly disappear.

In the present specification “comprise” means “includes or consists of” and “comprising” means “including or consisting of”.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof

### EXAMPLES

The present invention is described in more detail with reference to the following non-limiting examples, which are offered to more fully illustrate the invention, but are not to be construed as limiting the scope thereof

### Example 1

### Isolation and Preparation of Canine Plaque Samples

Supra-gingival plaque was collected from ten Labrador retrievers and ten miniature Schnauzers selected from a group of dogs undergoing weekly plaque collections. None of the dogs received tooth brushing and all were fed a variety of diets. Plaque samples were either collected prior to feeding or at least one hour after feeding. Supragingival plaque was collected from all of the teeth by scraping plastic loops (Appleton woods, UK) along the tooth surface. The plaque was placed in cryovials containing Ringers Solution (Oxoid). The samples were snap frozen in liquid nitrogen and stored at −80° C.

Nucleic Acid Extraction from Canine Plaque—

DNA and RNA was co-extracted from canine plaque samples (n=20) according to the hexadecyltrimethylammonium bromide (CTAB) and phenol/chloroform/isoamyl alcohol (25:24:1) extraction protocol of Griffiths et al. (30) and stored at −80° C. in nuclease free water.

Gel Extraction and Purification of Genomic DNA and Small-Subunit rRNA—

Nucleic acids extracted from canine plaque samples were pooled and visualised in 1% low melting point agarose (Sigma-Aldrich) gels following electrophoresis. Nucleic acids corresponding to genomic DNA (≧20 Kb) and Small-SubUnit rRNA (16S and 18S, ca. 1 Kb) were excised from the agarose gel for purification.

### Purification of Genomic DNA—

Genomic DNA was purified from the agarose gel slice using the QiaQuick Gel Extraction kit (Qiagen) following the manufacturer's protocol, and purified DNA was eluted into nuclease-free water and stored at −20° C. until required.

Purification of Small-Subunit rRNA—

SSU rRNA was purified from agarose gels using β-Agarase I (New England Biolabs) following the manufacturers' protocol with two modifications: 30 units of RNasin Plus Ribonuclease inhibitor (Promega) and 3 units of Turbo DNA-free (Ambion) were added. SSU rRNA was subsequently purified by precipitation with ¼ volume 10 M Ammonium Acetate and 2× vol. 100% ice-cold ethanol and incubated at −80° C. for 30 minutes. Following centrifugation at 13,000 rpm for 15 minutes, the RNA pellet was washed in 70% ethanol, resuspended in nuclease-free water and stored at −80° C. until required.

Reverse-Transcription of SSU rRNA into Double-Stranded cDNA—

two micrograms of gel extracted and purified SSU rRNA from canine plaque samples was reverse-transcribed using a Just cDNA™ Double-Stranded cDNA Synthesis Kit (Agilent Technologies) following the manufacturers' protocol and using random primers (9 mers, Agilent Technologies). Double-stranded cDNA was stored at −20° C. prior to library preparation for 454 pyrosequencing (Accession no: SRR830919).

16S rRNA Gene PCR Amplification of Canine Plaque DNA—

PCR reactions were performed in 50 μl volumes containing: 0.2 mM each primer V1-V3F 5′-GCCTAACACATGCAAGTC-3′ (SEQ ID NO: 1) (16) and V1-V3R 5′-ATTACCGCGGCTGCTGG-3′ (SEQ ID NO: 2) (17), 0.2 mM each dNTP, 1× Phusion HF buffer (Finnzymes), 0.5 units Phusion™ High-Fidelity DNA Polymerase (Finnzymes), 10 ng of pooled canine plaque DNA and ddH₂O. PCR cycling conditions were as follows; 98° C. for 45 s, 20 cycles of 98° C. for 10 s, 55° C. for 30 s and 72° C. for 15 s, and a final extension of 72° C. for 8 min. To minimise PCR bias, 20 cycles of amplification were performed in 8 separate replicate assays, and the PCR reactions were subsequently pooled. PCR amplification products were visualised using 1% agarose gel electrophoresis and fragments of the expected size (˜460 bp) were excised from the agarose gel and purified using a QiaQuick Gel Extraction kit (Qiagen) following the manufacturers' protocol. Gel extracted and purified V1-V3 16S rRNA gene amplification products were subsequently pooled and quantified using a Qubit™ fluorimeter (Invitrogen) and stored at −20° C. prior to library preparation for 454 pyrosequencing (Accession no: SRR830918).

### Library Preparation and 454 Pyrosequencing—

Fragment libraries for the GS FLX Titanium series were prepared using the PCR amplicons (Accession no: SRR830918) and RT-SSU rRNA (Accession no: SRR830919) according to the rapid library preparation method (Roche) and each library was sequenced on ¼ slide of a GS FLX plate.

### Example 2

### Isolation and Preparation of Biological Samples Using an Activated Charcoal Extraction Step

The activated charcoal is prepared as a ‘slurry’ in ddH₂O. First, 5.6 g of activated charcoal (Fisher Chemical #C/4040/53) were mixed thoroughly with 50 mL of ddH₂O. This initial slurry was then centrifuged at 4,000 rpm for 10 minutes. The supernatant (containing the charcoal particles too small or low in mass to pellet) was removed. The remaining activated charcoal (now containing particles that will pellet at 4,000 rpm for 10 minutes) was then resuspended in a further 50 mL of ddH₂O.

This activated charcoal slurry is used in the pre-treatment step (which can be shaken and vortexed prior to usage if stored). The pre-treatment step involved adding 200 uL of the activated charcoal ‘slurry’ to the isolated biological sample (which can contain a chaotropic agent). The activated charcoal was dispersed throughout the mixture by gentle inversion and the samples underwent slow rotation for 1 hr to allow the activated charcoal to bind to any production chemicals, natural ions, small biomolecules etc. originally in the collected water samples.

Following this activated charcoal pre-treatment step, the slurry was then centrifuged at 4,000 rpm for 10 minutes. The biological sample (which can contain a chaotropic agent) was then decanted carefully from the charcoal pellets into new tubes. Finally, the silica beads were added to facilitate DNA (and RNA if required) binding and the DNA (and RNA if required) extracted using the methods described herein.

Test extractions were carried out by contaminating test water samples with genomic DNA from Desulfovibrio alaskensis. The water samples used were: (a) produced water (from a North Sea oil platform outlet); and (b) a control of commercially bought DNA/RNA-free ddH₂O (to represent pure water with no additional minerals or production chemicals).

The previously extracted D. alaskensis genomic DNA had been prepared via the phenol-chloroform bead-beating and precipitation methodology of Griffiths et al.

The following qPCR data were derived from the DNA extracts and an assay specifically designed and targeted to the D. alaskensis dsrA gene:

| D. alaskensis genomic DNA | | qPCR |
|---|---|---|
| present in sample | Type of water sample | copy number |
| Yes | Produced Water Only | 0 |
| Yes | ddH₂O Only | 155,850 |
| Yes | Produced water + Charcoal | 106,250 |

Without being bound by theory, it is believed that these qPCR failures from produced water samples could have been due to either/both of: (a) the prevention/blocking of D. alaskensis DNA binding to silica due to the production chemicals, natural ions and biomolecules etc. present in produced water (and not present in ddH₂O); and/or (b) the co-extraction/concentration of qPCR assay inhibitors from the produced water (but not the ddH₂O) which prevented qPCR amplification from D. alaskensis DNA that had nevertheless been isolated.

In a further example, four North Sea produced water samples were split into two. Half underwent the aforementioned methods without an activated charcoal pre-treatment step (−AC step) and half with an activated charcoal pre-treatment step (+AC step). The DNA extracts were then contaminated with identical known-copy-number human mtDNA sequences to test PCR inhibition. The following qPCR data were derived via a qPCR assay specifically designed and targeted to human mtDNA:

| North Sea | | qPCR | qPCR |
|---|---|---|---|
| Sample | Additional DNA | (−AC step extrⁿ) | (+AC step extrⁿ) |
| 1 | Human mtDNA | 28,211 | 29,663 |
| 2 | Human mtDNA | 27,953 | 28,531 |
| 3 | Human mtDNA | 0 | 31,519 |
| 4 | Human mtDNA | 1,873 | 31,536 |

As the data demonstrates, the use of an activated charcoal pre-treatment step can avoid or reduce co-extracting or concentration of qPCR assay inhibitors in identical samples undergoing the +AC step method and the −AC step method.

### Example 3

### High Throughput Sequencing of Isolated and Prepared PCR Amplicon and SSU RT-RNA Samples

PCR amplicon and SSU RT-RNA query sequences were quality checked and classified against the RDP, Greengenes and Silva databases using the computer-implemented methods of the present invention, as well as the Qiime and RDP classifiers of the prior art.

### Qiime—

the QIIME software package (version 1.4.0) was used to analyse the sequences from the PCR dataset. Briefly, all sequences were de-multiplexed and quality filtered, and reads with a minimum identity of 97% were clustered into operational taxonomic units (OTU's). The most abundant sequences chosen to represent each OTU, and taxonomy was assigned with the Ribosomal Database Project (RDP) classifier (25), and SILVA (23), with a minimum confidence threshold of 80%.

### RDP Classifier—

Sequences from the PCR and SSU RT-RNA datasets were classified and compared using the command line version of the RDP classifier (version 2.5) using the default settings.

### BION-Meta—

utilises the computer-implemented methods of the present invention to create taxonomic overviews from raw sequence data, but with its own methods, as detailed above. Briefly, BION-meta cleans and de-replicates sequence reads, detects chimeras in PCR amplicon datasets, calculates similarities and projects these onto the taxonomy of a reference database. BION-meta handles low quality sequences well (ignores low quality regions without discarding the entire sequence read), can detect sequences with low similarity scores, can often differentiate species, works with non-amplicon data, installs from sources with a single line and is fast.

### Detection of Primer Mismatches in SSU RT-RNA Sequences—

Query sequences derived from the RT-SSU rRNA dataset were aligned against their best-matching database sequences, and the number of mismatches, insertions and deletions with the universal bacterial primer sets used to create the PCR amplicon library were determined for query sequence alignments that included the forward and/or reverse primer site. These values were mapped to a taxonomy overview and used to determine the ratio of total primer site mismatches, insertions and deletions detected within that taxon to the number of sequences within the taxon that possessed mismatches and insertions and/or deletions (indels) in the primer binding site.

It is possible to compare the identity and relative abundance of microbial taxa generated using the amplification-independent methods of the invention (e.g. RT-SSU eRNA direct sequencing) with those generated using the amplification-dependent methods (e.g. PCT amplicon sequencing) of the present invention. The comparison can involve simultaneously performing 454-pyrosequencing on a reverse-transcribed SSU rRNA (RT-SSU rRNA) library and a 16S rRNA gene PCR amplicon library generated from a single pooled canine plaque sample. The computer-implemented methods of the present invention can then process, quality check and classify the RT-SSU rRNA sequence reads generated.

For comparative analyses of the PCR amplicon and RT-SSU rRNA sequence output (248,760 and 257,043 sequence reads, respectively), the diversity and read densities of each dataset was examined using the computer-implemented methods of the present invention and the data benchmarked against the outputs of Qiime for the PCR amplicon library data or to the RDP classifier for the RT-SSU RNA dataset.

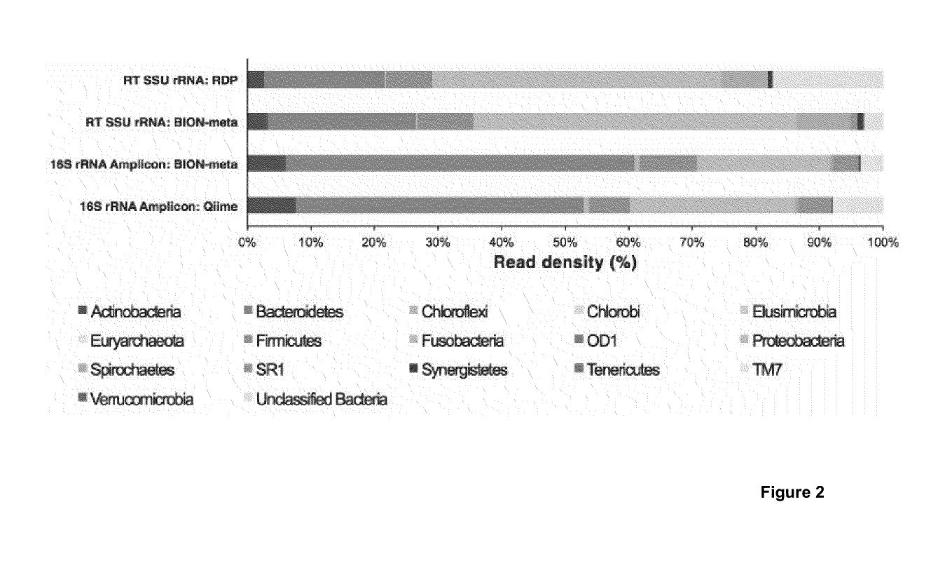

As shown in FIG. 2 and Tables 1 and 2 below, the computer implemented methods of the invention can be used to classify and compare sequence reads obtained from 16S rRNA gene PCR amplicons and RT-SSU rRNA from the same canine plaque sample. In order to compare the accuracy of the computer implemented method, the 16S rRNA amplicon dataset was also classified using Qiime and RT-SSU rRNA using the RDP classifier. The RDP database was used as a reference dataset for sequence classification.

The computer-implemented methods of the present invention provided similar classification data for both libraries compared to the widely used and validated programs Qiime and RDP classifier (FIG. 2 and Tables 1 and 2 below).

TABLE 1
Summary table of statistics for the processing and classification
of the sequence data presented in FIG. 2

| | 16S rRNA gene PCR | | RT-SSU rRNA | |
|---|---|---|---|---|
| | amplicons | | sequences | |
| | | BION- | RDP | BION- |
| | Qiime | meta | classifier | meta |
| Sequences in raw dataset | 248,760 | 248,760 | 257,043 | 257,043 |
| Sequences remaining | 149,185 | 177,424^{a} | 253,794 | 225,340^{a} |
| following QC | | | | |
| Sequences remaining | 153,866^{b} | 167,823 | N/A | 221,172 |
| following chimera | | | | |
| removal | | | | |
| Classified sequences | 67,682 | 99,998 | 127,113 | 100,009 |
| % sequences classified | 27% | 40% | 49% | 39% |

^{a}Most sequences are removed because they are short (<200 bp) or, some because they are low quality (90% of all positions must have at least 95% quality values) (See Supplementary Info: 16S PCR amplicon recipe). Please refer to supplementary description of BION-meta for more information.
^{b}The chimera removal step was completed prior to sequence QC, hence the higher number of sequences shown at this step.

TABLE 2
Classification of 16S rRNA gene PCR amplicons (Accession: SRR830918)
and RT-SSU rRNA sequence reads (Accession: SRR830919) using
the RDP classifier and library comparison tool.

| | | | PCR amplicons | | SSU RT-RNA | |
|---|---|---|---|---|---|---|
| | | | No. | | No. | |
| Significance | Rank | Taxon | reads | % total | reads | % total |
| 6.65E−09 | Phylum | Firmicutes | 10925 | 7.476 | 9300 | 7.316 |
| NA | | unclassified “Firmicutes” | 552 | 0.378 | 315 | 0.248 |
| 4.24E−03 | class | Bacilli | 495 | 0.339 | 545 | 0.429 |
| NA | | unclassified “Bacilli” | 7 | 0.005 | 12 | 0.009 |
| 6.40E−04 | order | Bacillales | 7 | 0.005 | 25 | 0.020 |
| NA | | unclassified Bacillales | 7 | 0.005 | 13 | 0.010 |
| 1.43E−04 | family | Staphylococcaceae | 0 | 0.000 | 12 | 0.009 |
| 1.43E−04 | genus | Gemella | 0 | 0.000 | 12 | 0.009 |
| 3.24E−02 | order | Lactobacillales | 481 | 0.329 | 508 | 0.400 |
| NA | | unclassified | 18 | 0.012 | 9 | 0.007 |
| | | “Lactobacillales” | | | | |
| 1.26E−12 | family | Aerococcaceae | 103 | 0.070 | 19 | 0.015 |
| NA | | unclassified | 46 | 0.031 | 7 | 0.006 |
| | | “Aerococcaceae” | | | | |
| 1.64E−03 | genus | Abiotrophia | 13 | 0.009 | 1 | 0.001 |
| 1.07E−07 | genus | Facklamia | 41 | 0.028 | 5 | 0.004 |
| 1.17E−02 | genus | Globicatella | 0 | 0.000 | 6 | 0.005 |
| 1.47E−01 | genus | Ignavigranum | 3 | 0.002 | 0 | 0.000 |
| 4.23E−06 | family | Carnobacteriaceae | 359 | 0.246 | 458 | 0.360 |
| NA | | unclassified | 4 | 0.003 | 5 | 0.004 |
| | | “Carnobacteriaceae” | | | | |
| 1.08E−05 | genus | Granulicatella | 355 | 0.243 | 449 | 0.353 |
| 5.08E−02 | genus | Trichococcus | 0 | 0.000 | 4 | 0.003 |
| 9.39E−01 | family | Enterococcaceae | 1 | 0.001 | 1 | 0.001 |
| NA | | unclassified | 1 | 0.001 | 0 | 0.000 |
| | | “Enterococcaceae” | | | | |
| 4.60E−01 | genus | Bavariicoccus | 0 | 0.000 | 1 | 0.001 |
| 1.92E−07 | family | Streptococcaceae | 0 | 0.000 | 21 | 0.017 |
| 1.92E−07 | genus | Streptococcus | 0 | 0.000 | 21 | 0.017 |
| 3.81E−08 | class | Clostridia | 9757 | 6.676 | 8305 | 6.534 |
| NA | | unclassified “Clostridia” | 110 | 0.075 | 244 | 0.192 |
| 1.05E−11 | order | Clostridiales | 9646 | 6.601 | 8051 | 6.334 |
| NA | | unclassified Clostridiales | 3382 | 2.314 | 2523 | 1.985 |
| 6.00E−14 | family | Veillonellaceae | 699 | 0.478 | 111 | 0.087 |
| NA | | unclassified | 180 | 0.123 | 30 | 0.024 |
| | | Veillonellaceae | | | | |
| 4.60E−01 | genus | Megamonas | 0 | 0.000 | 1 | 0.001 |
| 6.00E−14 | genus | Schwartzia | 500 | 0.342 | 70 | 0.055 |
| 1.47E−01 | genus | Succinispira | 3 | 0.002 | 0 | 0.000 |
| 2.90E−03 | genus | Succiniclasticum | 9 | 0.006 | 0 | 0.000 |
| 2.21E−01 | genus | Centipeda | 0 | 0.000 | 2 | 0.002 |
| 6.74E−01 | genus | Selenomonas | 7 | 0.005 | 8 | 0.006 |
| 6.00E−14 | family | Incertae Sedis XIII | 470 | 0.322 | 169 | 0.133 |
| NA | | unclassified Incertae Sedis | 6 | 0.004 | 7 | 0.006 |
| | | XIII | | | | |
| 6.00E−14 | genus | Anaerovorax | 464 | 0.318 | 160 | 0.126 |
| 2.21E−01 | genus | Mogibacterium | 0 | 0.000 | 2 | 0.002 |
| 6.00E−14 | family | Ruminococcaceae | 594 | 0.406 | 132 | 0.104 |
| NA | | unclassified | 397 | 0.272 | 41 | 0.032 |
| | | “Ruminococcaceae” | | | | |
| 3.97E−02 | genus | Ethanoligenens | 5 | 0.003 | 0 | 0.000 |
| 8.81E−01 | genus | Acetivibrio | 7 | 0.005 | 7 | 0.006 |
| 5.61E−03 | genus | Papillibacter | 0 | 0.000 | 7 | 0.006 |
| 2.59E−01 | genus | Fastidiosipila | 4 | 0.003 | 1 | 0.001 |
| 3.84E−20 | genus | Anaerotruncus | 74 | 0.051 | 1 | 0.001 |
| 6.65E−15 | genus | Lactonifactor | 3 | 0.002 | 56 | 0.044 |
| 6.00E−13 | genus | Oscillibacter | 104 | 0.071 | 19 | 0.015 |
| 8.07E−11 | family | Clostridiaceae | 238 | 0.163 | 376 | 0.296 |
| NA | | unclassified Clostridiaceae | 203 | 0.139 | 316 | 0.249 |
| 2.08E−01 | sub family | Clostridiaceae 4 | 4 | 0.003 | 8 | 0.006 |
| NA | | unclassified Clostridiaceae 4 | 4 | 0.003 | 7 | 0.006 |
| 4.60E−01 | genus | Thermotalea | 0 | 0.000 | 1 | 0.001 |
| 5.12E−03 | sub family | Clostridiaceae 2 | 30 | 0.021 | 52 | 0.041 |
| NA | | unclassified Clostridiaceae 2 | 27 | 0.018 | 34 | 0.027 |
| 1.58E−05 | genus | Tindallia | 0 | 0.000 | 15 | 0.012 |
| 9.12E−01 | genus | Anoxynatronum | 3 | 0.002 | 3 | 0.002 |
| 5.41E−01 | sub family | Clostridiaceae 3 | 1 | 0.001 | 0 | 0.000 |
| 5.41E−01 | genus | Clostridiisalibacter | 1 | 0.001 | 0 | 0.000 |
| 1.77E−01 | family | Peptococcaceae | 450 | 0.308 | 454 | 0.357 |
| NA | | unclassified | 2 | 0.001 | 3 | 0.002 |
| | | Peptococcaceae | | | | |
| 1.87E−01 | sub family | Peptococcaceae 1 | 448 | 0.307 | 451 | 0.355 |
| NA | | unclassified | 51 | 0.035 | 67 | 0.053 |
| | | Peptococcaceae 1 | | | | |
| 1.17E−02 | genus | Dehalobacter | 0 | 0.000 | 6 | 0.005 |
| 6.53E−01 | genus | Peptococcus | 397 | 0.272 | 378 | 0.297 |
| 1.06E−09 | family | Incertae Sedis XI | 431 | 0.295 | 585 | 0.460 |
| NA | | unclassified Incertae Sedis | 189 | 0.129 | 127 | 0.100 |
| | | XI | | | | |
| 7.62E−02 | genus | Finegoldia | 4 | 0.003 | 0 | 0.000 |
| 1.06E−01 | genus | Peptoniphilus | 0 | 0.000 | 3 | 0.002 |
| 3.62E−03 | genus | Parvimonas | 144 | 0.099 | 90 | 0.071 |
| 3.15E−12 | genus | Tissierella | 0 | 0.000 | 36 | 0.028 |
| 2.44E−02 | genus | Soehngenia | 0 | 0.000 | 5 | 0.004 |
| 6.64E−75 | genus | Helcococcus | 3 | 5.882 | 250 | 0.197 |
| 1.35E−02 | genus | Sporanaerobacter | 48 | 0.033 | 70 | 0.055 |
| 7.41E−09 | genus | Anaerosphaera | 43 | 0.029 | 4 | 0.003 |
| 6.20E−04 | family | Syntrophomonadaceae | 0 | 0.000 | 10 | 0.008 |
| 6.20E−04 | genus | Syntrophothermus | 0 | 0.000 | 10 | 0.008 |
| 5.48E−01 | family | Incertae Sedis XII | 484 | 0.331 | 429 | 0.337 |
| NA | | unclassified Incertae Sedis | 340 | 0.233 | 107 | 0.084 |
| | | XII | | | | |
| 2.21E−01 | genus | Acidaminobacter | 0 | 0.000 | 2 | 0.002 |
| 2.10E−11 | genus | Guggenheimella | 104 | 0.071 | 23 | 0.018 |
| 6.00E−14 | genus | Fusibacter | 40 | 0.027 | 297 | 0.234 |
| 6.74E−01 | family | Peptostreptococcaceae | 1082 | 0.740 | 1016 | 0.799 |
| NA | | unclassified | 19 | 0.013 | 182 | 0.143 |
| | | “Peptostreptococcaceae” | | | | |
| 2.69E−03 | genus | Tepidibacter | 0 | 0.000 | 8 | 0.006 |
| 4.13E−05 | genus | Filifactor | 1052 | 0.720 | 798 | 0.628 |
| 6.13E−01 | genus | Sporacetigenium | 2 | 0.001 | 3 | 0.002 |
| 2.88E−03 | genus | Peptostreptococcus | 9 | 0.006 | 25 | 0.020 |
| 6.00E−14 | family | Incertae Sedis XIV | 73 | 0.050 | 330 | 0.260 |
| NA | | unclassified Incertae Sedis | 5 | 0.003 | 18 | 0.014 |
| | | XIV | | | | |
| 3.26E−01 | genus | Anaerovirgula | 1 | 0.001 | 3 | 0.002 |
| 4.60E−01 | genus | Blautia | 0 | 0.000 | 1 | 0.001 |
| 6.00E−14 | genus | Proteocatella | 67 | 0.046 | 308 | 0.242 |
| 3.40E−07 | family | Lachnospiraceae | 1741 | 1.191 | 1901 | 1.496 |
| NA | | unclassified | 1348 | 0.922 | 1029 | 0.810 |
| | | “Lachnospiraceae” | | | | |
| 1.51E−03 | genus | Acetitomaculum | 10 | 0.007 | 0 | 0.000 |
| 6.67E−01 | genus | Oribacterium | 5 | 0.003 | 6 | 0.005 |
| 3.84E−01 | genus | Butyrivibrio | 58 | 0.040 | 45 | 0.035 |
| 8.57E−02 | genus | Coprococcus | 4 | 0.003 | 10 | 0.008 |
| 4.60E−01 | genus | Syntrophococcus | 0 | 0.000 | 1 | 0.001 |
| 6.00E−14 | genus | Catonella | 232 | 0.159 | 728 | 0.573 |
| 1.29E−03 | genus | Johnsonella | 0 | 0.000 | 9 | 0.007 |
| 2.83E−01 | genus | Dorea | 7 | 0.005 | 3 | 0.002 |
| 2.69E−03 | genus | Moryella | 0 | 0.000 | 8 | 0.006 |
| 1.79E−01 | genus | Hespellia | 3 | 0.002 | 7 | 0.006 |
| 9.39E−01 | genus | Shuttleworthia | 1 | 0.001 | 1 | 0.001 |
| 1.24E−01 | genus | Parasporobacterium | 9 | 0.006 | 3 | 0.002 |
| 3.97E−02 | genus | Anaerostipes | 5 | 0.003 | 0 | 0.000 |
| 8.57E−01 | genus | Robinsoniella | 34 | 0.023 | 30 | 0.024 |
| 7.49E−01 | genus | Anaerosporobacter | 25 | 0.017 | 21 | 0.017 |
| 7.27E−04 | family | Eubacteriaceae | 2 | 0.001 | 15 | 0.012 |
| NA | | unclassified | 2 | 0.001 | 4 | 0.003 |
| | | “Eubacteriaceae” | | | | |
| 1.17E−02 | genus | Alkalibacter | 0 | 0.000 | 6 | 0.005 |
| 2.44E−02 | genus | Eubacterium | 0 | 0.000 | 5 | 0.004 |
| 4.17E−03 | order | Thermoanaerobacterales | 1 | 0.001 | 10 | 0.008 |
| 4.17E−03 | family | Thermoanaerobacteraceae | 1 | 0.001 | 10 | 0.008 |
| NA | | unclassified | 0 | 0.000 | 3 | 0.002 |
| | | “Thermoanaerobacteraceae” | | | | |
| 4.60E−01 | genus | Gelria | 0 | 0.000 | 1 | 0.001 |
| 1.17E−02 | genus | Mahella | 0 | 0.000 | 6 | 0.005 |
| 5.41E−01 | genus | Thermovenabulum | 1 | 0.001 | 0 | 0.000 |
| 1.29E−01 | class | Erysipelotrichi | 121 | 0.083 | 135 | 0.106 |
| 1.29E−01 | order | Erysipelotrichales | 121 | 0.083 | 135 | 0.106 |
| 1.29E−01 | family | Erysipelotrichaceae | 121 | 0.083 | 135 | 0.106 |
| NA | | unclassified | 105 | 0.072 | 35 | 0.028 |
| | | Erysipelotrichaceae | | | | |
| 8.75E−02 | genus | Allobaculum | 8 | 0.005 | 2 | 0.002 |
| 2.21E−01 | genus | Bulleidia | 0 | 0.000 | 2 | 0.002 |
| 5.32E−24 | genus | Holdemania | 1 | 0.001 | 78 | 0.061 |
| 1.64E−02 | genus | Solobacterium | 7 | 0.005 | 18 | 0.014 |
| 6.00E−14 | Phylum | Actinobacteria | 9592 | 6.564 | 3390 | 2.667 |
| 6.00E−14 | class | Actinobacteria | 9592 | 6.564 | 3390 | 2.667 |
| NA | | unclassified | 4 | 0.003 | 1 | 0.001 |
| | | Actinobacteria | | | | |
| 6.00E−14 | sub class | Actinobacteridae | 9588 | 6.561 | 3389 | 2.666 |
| NA | | unclassified | 12 | 0.008 | 17 | 0.013 |
| | | Actinobacteridae | | | | |
| 6.00E−14 | order | Actinomycetales | 9576 | 6.553 | 3372 | 2.653 |
| NA | | unclassified | 745 | 0.510 | 119 | 0.094 |
| | | Actinomycetales | | | | |
| 6.00E−14 | sub order | Actinomycineae | 4483 | 3.068 | 1533 | 1.206 |
| 6.00E−14 | family | Actinomycetaceae | 4483 | 3.068 | 1533 | 1.206 |
| NA | | unclassified | 102 | 0.070 | 19 | 0.015 |
| | | Actinomycetaceae | | | | |
| 6.00E−14 | genus | Actinomyces | 4378 | 2.996 | 1514 | 1.191 |
| 1.47E−01 | genus | Varibaculum | 3 | 0.002 | 0 | 0.000 |
| 6.00E−14 | sub order | Corynebacterineae | 2364 | 1.618 | 1189 | 0.935 |
| NA | | unclassified | 61 | 0.042 | 12 | 0.009 |
| | | Corynebacterineae | | | | |
| 6.00E−14 | family | Corynebacteriaceae | 2303 | 1.576 | 1175 | 0.924 |
| NA | | unclassified | 50 | 0.034 | 11 | 0.009 |
| | | Corynebacteriaceae | | | | |
| 6.00E−14 | genus | Corynebacterium | 2251 | 1.540 | 1163 | 0.915 |
| 6.87E−01 | genus | Turicella | 2 | 0.001 | 1 | 0.001 |
| 2.21E−01 | family | Nocardiaceae | 0 | 0.000 | 2 | 0.002 |
| 2.21E−01 | genus | Millisia | 0 | 0.000 | 2 | 0.002 |
| 1.06E−01 | sub order | Kineosporiineae | 0 | 0.000 | 3 | 0.002 |
| 1.06E−01 | family | Kineosporiaceae | 0 | 0.000 | 3 | 0.002 |
| NA | | unclassified | 0 | 0.000 | 2 | 0.002 |
| | | Kineosporiaceae | | | | |
| 4.60E−01 | genus | Kineococcus | 0 | 0.000 | 1 | 0.001 |
| 6.00E−14 | sub order | Micrococcineae | 1703 | 1.165 | 379 | 0.298 |
| NA | | unclassified | 582 | 0.398 | 63 | 0.050 |
| | | Micrococcineae | | | | |
| 5.41E−01 | family | Beutenbergiaceae | 1 | 0.001 | 0 | 0.000 |
| NA | | unclassified | 1 | 0.001 | 0 | 0.000 |
| | | Beutenbergiaceae | | | | |
| 1.83E−01 | family | Cellulomonadaceae | 1 | 0.001 | 4 | 0.003 |
| NA | | unclassified | 0 | 0.000 | 4 | 0.003 |
| | | Cellulomonadaceae | | | | |
| 5.41E−01 | genus | Cellulomonas | 1 | 0.001 | 0 | 0.000 |
| 4.60E−01 | family | Dermabacteraceae | 0 | 0.000 | 1 | 0.001 |
| 4.60E−01 | genus | Devriesea | 0 | 0.000 | 1 | 0.001 |
| 5.19E−02 | family | Intrasporangiaceae | 7 | 0.005 | 1 | 0.001 |
| NA | | unclassified | 4 | 0.003 | 0 | 0.000 |
| | | Intrasporangiaceae | | | | |
| 4.28E−01 | genus | Marihabitans | 3 | 0.002 | 1 | 0.001 |
| 9.31E−02 | family | Jonesiaceae | 13 | 0.009 | 5 | 0.004 |
| 9.31E−02 | genus | Jonesia | 13 | 0.009 | 5 | 0.004 |
| 6.00E−14 | family | Microbacteriaceae | 1094 | 0.749 | 305 | 0.240 |
| NA | | unclassified | 855 | 0.585 | 192 | 0.151 |
| | | Microbacteriaceae | | | | |
| 5.41E−01 | genus | Agrococcus | 1 | 0.001 | 0 | 0.000 |
| 5.41E−01 | genus | Clavibacter | 1 | 0.001 | 0 | 0.000 |
| 1.12E−01 | genus | Curtobacterium | 6 | 0.004 | 12 | 0.009 |
| 7.62E−02 | genus | Humibacter | 4 | 0.003 | 0 | 0.000 |
| 9.39E−01 | genus | Klugiella | 1 | 0.001 | 1 | 0.001 |
| 9.60E−07 | genus | Leucobacter | 156 | 0.107 | 72 | 0.057 |
| 4.60E−01 | genus | Plantibacter | 0 | 0.000 | 1 | 0.001 |
| 5.08E−02 | genus | Pseudoclavibacter | 0 | 0.000 | 4 | 0.003 |
| 7.14E−15 | genus | Rathayibacter | 55 | 0.038 | 1 | 0.001 |
| 4.60E−01 | genus | Subtercola | 0 | 0.000 | 1 | 0.001 |
| 2.15E−01 | genus | Zimmermannella | 15 | 0.010 | 21 | 0.017 |
| 3.97E−02 | family | Micrococcineae_incertae_sedis | 5 | 0.003 | 0 | 0.000 |
| 3.97E−02 | genus | Ruania | 5 | 0.003 | 0 | 0.000 |
| 3.81E−08 | sub order | Propionibacterineae | 281 | 0.192 | 149 | 0.117 |
| NA | | unclassified | 0 | 0.000 | 1 | 0.001 |
| | | Propionibacterineae | | | | |
| 3.81E−08 | family | Propionibacteriaceae | 281 | 0.192 | 148 | 0.116 |
| NA | | unclassified | 186 | 0.127 | 74 | 0.058 |
| | | Propionibacteriaceae | | | | |
| 1.15E−02 | genus | Aestuariimicrobium | 12 | 0.008 | 2 | 0.002 |
| 5.22E−01 | genus | Brooklawnia | 4 | 0.003 | 2 | 0.002 |
| 4.13E−05 | genus | Luteococcus | 57 | 0.039 | 18 | 0.014 |
| 8.37E−02 | genus | Propionibacterium | 2 | 0.001 | 7 | 0.006 |
| 2.21E−01 | genus | Propioniferax | 0 | 0.000 | 2 | 0.002 |
| 1.28E−03 | genus | Tessaracoccus | 20 | 0.014 | 43 | 0.034 |
| 6.00E−14 | Phylum | Bacteroidetes | 85560 | 58.547 | 23975 | 18.861 |
| NA | | unclassified | 1951 | 1.335 | 2070 | 1.628 |
| | | “Bacteroidetes” | | | | |
| 6.00E−14 | class | Bacteroidia | 80670 | 55.200 | 14505 | 11.411 |
| 6.00E−14 | order | Bacteroidales | 80670 | 55.200 | 14505 | 11.411 |
| NA | | unclassified | 965 | 0.660 | 829 | 0.652 |
| | | “Bacteroidales” | | | | |
| 1.00E−03 | family | Bacteroidaceae | 742 | 0.508 | 570 | 0.448 |
| 1.00E−03 | genus | Bacteroides | 742 | 0.508 | 570 | 0.448 |
| 2.21E−01 | family | Marinilabiaceae | 0 | 0.000 | 2 | 0.002 |
| 2.21E−01 | genus | Anaerophaga | 0 | 0.000 | 2 | 0.002 |
| 6.00E−14 | family | Porphyromonadaceae | 77704 | 53.171 | 12134 | 9.546 |
| NA | | unclassified | 3835 | 2.624 | 646 | 0.508 |
| | | “Porphyromonadaceae” | | | | |
| 3.35E−04 | genus | Dysgonomonas | 21 | 0.014 | 3 | 0.002 |
| 3.22E−01 | genus | Odoribacter | 20 | 0.014 | 13 | 0.010 |
| 2.26E−01 | genus | Paludibacter | 110 | 0.075 | 119 | 0.094 |
| 6.00E−14 | genus | Parabacteroides | 328 | 0.224 | 107 | 0.084 |
| 2.00E−07 | genus | Petrimonas | 61 | 0.042 | 13 | 0.010 |
| 6.00E−14 | genus | Porphyromonas | 72270 | 49.453 | 10605 | 8.343 |
| 8.07E−11 | genus | Proteiniphilum | 137 | 0.094 | 42 | 0.033 |
| 1.26E−12 | genus | Tannerella | 922 | 0.631 | 586 | 0.461 |
| 2.60E−06 | family | Prevotellaceae | 1259 | 0.862 | 948 | 0.746 |
| NA | | unclassified | 593 | 0.406 | 440 | 0.346 |
| | | “Prevotellaceae” | | | | |
| 6.00E−14 | genus | Hallella | 212 | 0.145 | 71 | 0.056 |
| 6.00E−14 | genus | Paraprevotella | 41 | 0.028 | 326 | 0.256 |
| 6.00E−14 | genus | Prevotella | 412 | 0.282 | 107 | 0.084 |
| 1.83E−01 | genus | Xylanibacter | 1 | 0.001 | 4 | 0.003 |
| 9.22E−08 | family | Rikenellaceae | 0 | 0.000 | 22 | 0.017 |
| 9.22E−08 | genus | Rikenella | 0 | 0.000 | 22 | 0.017 |
| 6.00E−14 | class | Flavobacteria | 2753 | 1.884 | 7279 | 5.726 |
| 6.00E−14 | order | Flavobacteriales | 2753 | 1.884 | 7279 | 5.726 |
| NA | | unclassified | 34 | 0.023 | 18 | 0.014 |
| | | “Flavobacteriales” | | | | |
| 6.00E−14 | family | Flavobacteriaceae | 2719 | 1.861 | 7261 | 5.712 |
| NA | | unclassified | 425 | 0.291 | 465 | 0.366 |
| | | Flavobacteriaceae | | | | |
| 2.21E−01 | genus | Aquimarina | 0 | 0.000 | 2 | 0.002 |
| 6.00E−14 | genus | Capnocytophaga | 857 | 0.586 | 5371 | 4.225 |
| 2.24E−53 | genus | Chryseobacterium | 0 | 0.000 | 165 | 0.130 |
| 4.13E−05 | genus | Cloacibacterium | 52 | 0.036 | 15 | 0.012 |
| 5.08E−02 | genus | Coenonia | 0 | 0.000 | 4 | 0.003 |
| 4.60E−01 | genus | Croceibacter | 0 | 0.000 | 1 | 0.001 |
| 2.82E−01 | genus | Epilithonimonas | 2 | 0.001 | 0 | 0.000 |
| 6.00E−14 | genus | Flavobacterium | 755 | 0.517 | 202 | 0.159 |
| 1.83E−01 | genus | Kaistella | 1 | 0.001 | 4 | 0.003 |
| 6.00E−14 | genus | Riemerella | 508 | 0.348 | 1016 | 0.799 |
| 3.14E−28 | genus | Myroides | 117 | 0.080 | 4 | 0.003 |
| 4.62E−03 | genus | Planobacterium | 2 | 0.001 | 12 | 0.009 |
| 1.74E−03 | class | Sphingobacteria | 186 | 0.127 | 119 | 0.094 |
| 1.74E−03 | order | Sphingobacteriales | 186 | 0.127 | 119 | 0.094 |
| NA | | unclassified | 79 | 0.054 | 71 | 0.056 |
| | | “Sphingobacteriales” | | | | |
| 1.06E−01 | family | Cytophagaceae | 0 | 0.000 | 3 | 0.002 |
| NA | | unclassified | 0 | 0.000 | 3 | 0.002 |
| | | Cytophagaceae | | | | |
| 6.72E−02 | family | Flammeovirgaceae | 20 | 0.014 | 9 | 0.007 |
| NA | | unclassified | 20 | 0.014 | 9 | 0.007 |
| | | “Flammeovirgaceae” | | | | |
| 4.13E−05 | family | Sphingobacteriaceae | 87 | 0.060 | 36 | 0.028 |
| NA | | unclassified | 4 | 0.003 | 6 | 0.005 |
| | | Sphingobacteriaceae | | | | |
| 6.80E−06 | genus | Nubsella | 83 | 0.057 | 30 | 0.024 |
| 2.21E−01 | family | Bacteroidetes_incertae_sedis | 0 | 0.000 | 2 | 0.002 |
| 2.21E−01 | genus | Prolixibacter | 0 | 0.000 | 2 | 0.002 |
| 6.00E−14 | Phylum | Chloroflexi | 969 | 0.663 | 270 | 0.212 |
| NA | | unclassified “Chloroflexi” | 7 | 0.005 | 0 | 0.000 |
| 6.00E−14 | class | Anaerolineae | 962 | 0.658 | 270 | 0.212 |
| 6.00E−14 | order | Anaerolineales | 962 | 0.658 | 270 | 0.212 |
| 6.00E−14 | family | Anaerolineaceae | 962 | 0.658 | 270 | 0.212 |
| NA | | unclassified | 679 | 0.465 | 219 | 0.172 |
| | | Anaerolineaceae | | | | |
| 1.50E−07 | genus | Anaerolinea | 2 | 0.001 | 28 | 0.022 |
| 5.51E−10 | genus | Bellilinea | 41 | 0.028 | 2 | 0.002 |
| 2.21E−01 | genus | Leptolinea | 0 | 0.000 | 2 | 0.002 |
| 6.00E−14 | genus | Levilinea | 240 | 0.164 | 16 | 0.013 |
| 1.06E−01 | genus | Longilinea | 0 | 0.000 | 3 | 0.002 |
| 6.00E−14 | Phylum | Proteobacteria | 32255 | 22.071 | 57470 | 45.212 |
| NA | | unclassified | 696 | 0.476 | 545 | 0.429 |
| | | “Proteobacteria” | | | | |
| 1.68E−01 | class | Alphaproteobacteria | 45 | 0.031 | 30 | 0.024 |
| NA | | unclassified | 4 | 0.003 | 20 | 0.016 |
| | | Alphaproteobacteria | | | | |
| 1.14E−06 | order | Caulobacterales | 21 | 0.014 | 0 | 0.000 |
| 1.14E−06 | family | Caulobacteraceae | 21 | 0.014 | 0 | 0.000 |
| NA | | unclassified | 2 | 0.001 | 0 | 0.000 |
| | | Caulobacteraceae | | | | |
| 8.11E−06 | genus | Brevundimonas | 18 | 0.012 | 0 | 0.000 |
| 5.41E−01 | genus | Caulobacter | 1 | 0.001 | 0 | 0.000 |
| 1.07E−01 | order | Rhizobiales | 20 | 0.014 | 10 | 0.008 |
| NA | | unclassified Rhizobiales | 0 | 0.000 | 5 | 0.004 |
| 1.11E−04 | family | Brucellaceae | 14 | 0.010 | 0 | 0.000 |
| 1.11E−04 | genus | Ochrobactrum | 14 | 0.010 | 0 | 0.000 |
| 2.44E−02 | family | Hyphomicrobiaceae | 0 | 0.000 | 5 | 0.004 |
| NA | | unclassified | 0 | 0.000 | 2 | 0.002 |
| | | Hyphomicrobiaceae | | | | |
| 2.21E−01 | genus | Maritalea | 0 | 0.000 | 2 | 0.002 |
| 4.60E−01 | genus | Zhangella | 0 | 0.000 | 1 | 0.001 |
| 2.06E−02 | family | Phyllobacteriaceae | 6 | 0.004 | 0 | 0.000 |
| 2.06E−02 | genus | Defluvibacter | 6 | 0.004 | 0 | 0.000 |
| 6.00E−14 | class | Betaproteobacteria | 9047 | 6.191 | 19567 | 15.393 |
| NA | | unclassified | 131 | 0.090 | 179 | 0.141 |
| | | Betaproteobacteria | | | | |
| 6.00E−14 | order | Burkholderiales | 6464 | 4.423 | 13811 | 10.865 |
| NA | | unclassified | 499 | 0.341 | 845 | 0.665 |
| | | Burkholderiales | | | | |
| 6.00E−14 | family | Alcaligenaceae | 687 | 0.470 | 395 | 0.311 |
| NA | | unclassified | 222 | 0.152 | 169 | 0.133 |
| | | Alcaligenaceae | | | | |
| 2.06E−02 | genus | Achromobacter | 6 | 0.004 | 0 | 0.000 |
| 5.41E−01 | genus | Advenella | 1 | 0.001 | 0 | 0.000 |
| 5.41E−01 | genus | Alcaligenes | 1 | 0.001 | 0 | 0.000 |
| 1.17E−02 | genus | Bordetella | 0 | 0.000 | 6 | 0.005 |
| 6.00E−14 | genus | Castellaniella | 153 | 0.105 | 13 | 0.010 |
| 2.21E−01 | genus | Derxia | 0 | 0.000 | 2 | 0.002 |
| 1.47E−01 | genus | Kerstersia | 3 | 0.002 | 0 | 0.000 |
| 4.28E−01 | genus | Pelistega | 3 | 0.002 | 1 | 0.001 |
| 2.00E−07 | genus | Pigmentiphaga | 109 | 0.075 | 39 | 0.031 |
| 4.00E−07 | genus | Taylorella | 0 | 0.000 | 20 | 0.016 |
| 9.49E−02 | genus | Tetrathiobacter | 189 | 0.129 | 145 | 0.114 |
| 3.81E−08 | family | Burkholderiaceae | 91 | 0.062 | 26 | 0.020 |
| NA | | unclassified | 77 | 0.053 | 17 | 0.013 |
| | | Burkholderiaceae | | | | |
| 2.82E−01 | genus | Chitinimonas | 2 | 0.001 | 0 | 0.000 |
| 6.38E−01 | genus | Pandoraea | 12 | 0.008 | 9 | 0.007 |
| 6.00E−14 | family | Comamonadaceae | 5167 | 3.536 | 12443 | 9.789 |
| NA | | unclassified | 2097 | 1.435 | 4425 | 3.481 |
| | | Comamonadaceae | | | | |
| 5.43E−02 | genus | Acidovorax | 1 | 0.001 | 6 | 0.005 |
| 1.06E−01 | genus | Alicycliphilus | 0 | 0.000 | 3 | 0.002 |
| 6.00E−14 | genus | Brachymonas | 1108 | 0.758 | 174 | 0.137 |
| 6.00E−14 | genus | Comamonas | 1105 | 0.756 | 3733 | 2.937 |
| 1.06E−01 | genus | Delftia | 0 | 0.000 | 3 | 0.002 |
| 8.63E−74 | genus | Diaphorobacter | 0 | 0.000 | 229 | 0.180 |
| 3.15E−12 | genus | Hydrogenophaga | 0 | 0.000 | 36 | 0.028 |
| 2.82E−01 | genus | Hylemonella | 2 | 0.001 | 0 | 0.000 |
| 6.00E−14 | genus | Lampropedia | 466 | 0.319 | 2200 | 1.731 |
| 6.00E−14 | genus | Ottowia | 208 | 0.142 | 40 | 0.031 |
| 2.21E−01 | genus | Pseudacidovorax | 0 | 0.000 | 2 | 0.002 |
| 1.77E−21 | genus | Pseudorhodoferax | 0 | 0.000 | 65 | 0.051 |
| 5.74E−07 | genus | Schlegelella | 92 | 0.063 | 31 | 0.024 |
| 1.06E−01 | genus | Giesbergeria | 0 | 0.000 | 3 | 0.002 |
| 1.17E−02 | genus | Simplicispira | 0 | 0.000 | 6 | 0.005 |
| 4.00E−07 | genus | Tepidicella | 0 | 0.000 | 20 | 0.016 |
| 0.00E+00 | genus | Variovorax | 0 | 0.000 | 1406 | 1.106 |
| 8.54E−02 | genus | Xenophilus | 88 | 0.060 | 61 | 0.048 |
| 2.91E−26 | family | Oxalobacteraceae | 0 | 0.000 | 80 | 0.063 |
| NA | | unclassified | 0 | 0.000 | 78 | 0.061 |
| | | Oxalobacteraceae | | | | |
| 2.21E−01 | genus | Herbaspirillum | 0 | 0.000 | 2 | 0.002 |
| 5.69E−01 | family | Burkholderiales_incertae_sedis | 20 | 0.014 | 22 | 0.017 |
| NA | | unclassified | 13 | 0.009 | 9 | 0.007 |
| | | Burkholderiales_incertae_sedis | | | | |
| 8.81E−01 | genus | Tepidimonas | 7 | 0.005 | 7 | 0.006 |
| 1.17E−02 | genus | Thiomonas | 0 | 0.000 | 6 | 0.005 |
| 6.00E−14 | order | Neisseriales | 2192 | 1.500 | 5427 | 4.269 |
| 6.00E−14 | family | Neisseriaceae | 2192 | 1.500 | 5427 | 4.269 |
| NA | | unclassified Neisseriaceae | 601 | 0.411 | 1765 | 1.389 |
| 6.00E−14 | genus | Alysiella | 24 | 0.016 | 315 | 0.248 |
| 0.00E+00 | genus | Aquaspirillum | 0 | 0.000 | 1481 | 1.165 |
| 4.60E−01 | genus | Aquitalea | 0 | 0.000 | 1 | 0.001 |
| 5.61E−03 | genus | Bergeriella | 0 | 0.000 | 7 | 0.006 |
| 1.06E−01 | genus | Chitinibacter | 0 | 0.000 | 3 | 0.002 |
| 5.74E−07 | genus | Conchiformibius | 56 | 0.038 | 115 | 0.090 |
| 3.85E−14 | genus | Formivibrio | 0 | 0.000 | 42 | 0.033 |
| 2.58E−12 | genus | Kingella | 16 | 0.011 | 82 | 0.065 |
| 6.34E−05 | genus | Neisseria | 1479 | 1.012 | 1580 | 1.243 |
| 1.87E−01 | genus | Paludibacterium | 7 | 0.005 | 12 | 0.009 |
| 8.65E−01 | genus | Uruburuella | 9 | 0.006 | 9 | 0.007 |
| 1.58E−05 | genus | Vogesella | 0 | 0.000 | 15 | 0.012 |
| 4.23E−06 | order | Rhodocyclales | 260 | 0.178 | 150 | 0.118 |
| 4.23E−06 | family | Rhodocyclaceae | 260 | 0.178 | 150 | 0.118 |
| NA | | unclassified | 43 | 0.029 | 28 | 0.022 |
| | | Rhodocyclaceae | | | | |
| 3.09E−17 | genus | Azospira | 68 | 0.047 | 2 | 0.002 |
| 1.94E−01 | genus | Propionivibrio | 149 | 0.102 | 117 | 0.092 |
| 1.06E−01 | genus | Uliginosibacterium | 0 | 0.000 | 3 | 0.002 |
| 6.00E−14 | class | Deltaproteobacteria | 3262 | 2.232 | 1380 | 1.086 |
| NA | | unclassified | 172 | 0.118 | 145 | 0.114 |
| | | Deltaproteobacteria | | | | |
| 2.58E−12 | order | Bdellovibrionales | 320 | 0.219 | 149 | 0.117 |
| NA | | unclassified | 0 | 0.000 | 2 | 0.002 |
| | | Bdellovibrionales | | | | |
| 6.20E−04 | family | Bacteriovoracaceae | 0 | 0.000 | 10 | 0.008 |
| NA | | unclassified | 0 | 0.000 | 4 | 0.003 |
| | | Bacteriovoracaceae | | | | |
| 1.06E−01 | genus | Bacteriovorax | 0 | 0.000 | 3 | 0.002 |
| 1.06E−01 | genus | Peredibacter | 0 | 0.000 | 3 | 0.002 |
| 6.00E−14 | family | Bdellovibrionaceae | 320 | 0.219 | 137 | 0.108 |
| 6.00E−14 | genus | Bdellovibrio | 320 | 0.219 | 137 | 0.108 |
| 2.60E−06 | order | Desulfobacterales | 53 | 0.036 | 12 | 0.009 |
| NA | | unclassified | 1 | 0.001 | 0 | 0.000 |
| | | Desulfobacterales | | | | |
| 4.23E−06 | family | Desulfobulbaceae | 52 | 0.036 | 12 | 0.009 |
| 1.59E−06 | genus | Desulfobulbus | 52 | 0.036 | 11 | 0.009 |
| 4.60E−01 | genus | Desulfurivibrio | 0 | 0.000 | 1 | 0.001 |
| 6.00E−14 | order | Desulfovibrionales | 2702 | 1.849 | 1020 | 0.802 |
| NA | | unclassified | 64 | 0.044 | 11 | 0.009 |
| | | Desulfovibrionales | | | | |
| 9.39E−01 | family | Desulfohalobiaceae | 1 | 0.001 | 1 | 0.001 |
| NA | | unclassified | 1 | 0.001 | 1 | 0.001 |
| | | Desulfohalobiaceae | | | | |
| 6.00E−14 | family | Desulfomicrobiaceae | 2201 | 1.506 | 879 | 0.692 |
| 6.00E−14 | genus | Desulfomicrobium | 2201 | 1.506 | 879 | 0.692 |
| 6.00E−14 | family | Desulfovibrionaceae | 436 | 0.298 | 129 | 0.101 |
| NA | | unclassified | 113 | 0.077 | 7 | 0.006 |
| | | Desulfovibrionaceae | | | | |
| 6.00E−14 | genus | Desulfovibrio | 323 | 0.221 | 119 | 0.094 |
| 1.06E−01 | genus | Lawsonia | 0 | 0.000 | 3 | 0.002 |
| 5.74E−07 | order | Myxococcales | 15 | 0.010 | 54 | 0.042 |
| NA | | unclassified Myxococcales | 1 | 0.001 | 2 | 0.002 |
| 5.74E−07 | sub order | Sorangiineae | 14 | 0.010 | 52 | 0.041 |
| NA | | unclassified Sorangiineae | 0 | 0.000 | 2 | 0.002 |
| 1.59E−06 | family | Polyangiaceae | 14 | 0.010 | 50 | 0.039 |
| NA | | unclassified Polyangiaceae | 10 | 0.007 | 26 | 0.020 |
| 3.28E−05 | genus | Byssovorax | 0 | 0.000 | 14 | 0.011 |
| 1.35E−01 | genus | Chondromyces | 4 | 0.003 | 9 | 0.007 |
| 4.60E−01 | genus | Sorangium | 0 | 0.000 | 1 | 0.001 |
| 6.00E−14 | class | Epsilonproteobacteria | 635 | 0.435 | 3974 | 3.126 |
| NA | | unclassified | 6 | 0.004 | 12 | 0.009 |
| | | Epsilonproteobacteria | | | | |
| 6.00E−14 | order | Campylobacterales | 629 | 0.430 | 3959 | 3.115 |
| NA | | unclassified | 5 | 0.003 | 22 | 0.017 |
| | | Campylobacterales | | | | |
| 6.00E−14 | family | Campylobacteraceae | 219 | 0.150 | 3577 | 2.814 |
| NA | | unclassified | 0 | 0.000 | 2 | 0.002 |
| | | Campylobacteraceae | | | | |
| 6.00E−14 | genus | Arcobacter | 67 | 0.046 | 1583 | 1.245 |
| 6.00E−14 | genus | Campylobacter | 152 | 0.104 | 1992 | 1.567 |
| 5.69E−01 | family | Helicobacteraceae | 399 | 0.273 | 353 | 0.278 |
| NA | | unclassified | 5 | 0.003 | 8 | 0.006 |
| | | Helicobacteraceae | | | | |
| 1.74E−01 | genus | Helicobacter | 17 | 0.012 | 9 | 0.007 |
| 6.46E−01 | genus | Wolinella | 377 | 0.258 | 336 | 0.264 |
| 6.74E−01 | family | Hydrogenimonaceae | 6 | 0.004 | 7 | 0.006 |
| 6.74E−01 | genus | Hydrogenimonas | 6 | 0.004 | 7 | 0.006 |
| 1.06E−01 | order | Nautiliales | 0 | 0.000 | 3 | 0.002 |
| 1.06E−01 | family | Nautiliaceae | 0 | 0.000 | 3 | 0.002 |
| 1.06E−01 | genus | Thioreductor | 0 | 0.000 | 3 | 0.002 |
| 6.00E−14 | class | Gammaproteobacteria | 18570 | 12.707 | 31974 | 25.154 |
| NA | | unclassified | 1298 | 0.888 | 565 | 0.444 |
| | | Gammaproteobacteria | | | | |
| 6.00E−14 | order | Cardiobacteriales | 469 | 0.321 | 753 | 0.592 |
| 6.00E−14 | family | Cardiobacteriaceae | 469 | 0.321 | 753 | 0.592 |
| NA | | unclassified | 371 | 0.254 | 94 | 0.074 |
| | | Cardiobacteriaceae | | | | |
| 6.00E−14 | genus | Cardiobacterium | 59 | 0.040 | 242 | 0.190 |
| 4.60E−01 | genus | Dichelobacter | 0 | 0.000 | 1 | 0.001 |
| 6.00E−14 | genus | Suttonella | 39 | 0.027 | 416 | 0.327 |
| 3.23E−13 | order | Chromatiales | 49 | 0.034 | 1 | 0.001 |
| NA | | unclassified Chromatiales | 19 | 0.013 | 1 | 0.001 |
| 6.13E−09 | family | Ectothiorhodospiraceae | 29 | 0.020 | 0 | 0.000 |
| NA | | unclassified | 29 | 0.020 | 0 | 0.000 |
| | | Ectothiorhodospiraceae | | | | |
| 5.41E−01 | family | Halothiobacillaceae | 1 | 0.001 | 0 | 0.000 |
| NA | | unclassified | 1 | 0.001 | 0 | 0.000 |
| | | Halothiobacillaceae | | | | |
| 5.41E−01 | order | Enterobacteriales | 1 | 0.001 | 0 | 0.000 |
| 5.41E−01 | family | Enterobacteriaceae | 1 | 0.001 | 0 | 0.000 |
| 5.41E−01 | genus | Obesumbacterium | 1 | 0.001 | 0 | 0.000 |
| 2.04E−05 | order | Oceanospirillales | 3 | 0.002 | 23 | 0.018 |
| NA | | unclassified | 3 | 0.002 | 19 | 0.015 |
| | | Oceanospirillales | | | | |
| 5.08E−02 | family | Halomonadaceae | 0 | 0.000 | 4 | 0.003 |
| NA | | unclassified | 0 | 0.000 | 4 | 0.003 |
| | | Halomonadaceae | | | | |
| 6.00E−14 | order | Pasteurellales | 4268 | 2.920 | 1395 | 1.097 |
| 6.00E−14 | family | Pasteurellaceae | 4268 | 2.920 | 1395 | 1.097 |
| NA | | unclassified | 629 | 0.430 | 287 | 0.226 |
| | | Pasteurellaceae | | | | |
| 1.36E−01 | genus | Actinobacillus | 523 | 0.358 | 438 | 0.345 |
| 1.33E−03 | genus | Aggregatibacter | 16 | 0.011 | 2 | 0.002 |
| 6.00E−14 | genus | Bibersteinia | 242 | 0.166 | 32 | 0.025 |
| 6.00E−14 | genus | Haemophilus | 889 | 0.608 | 270 | 0.212 |
| 1.41E−01 | genus | Lonepinella | 7 | 0.005 | 2 | 0.002 |
| 5.08E−02 | genus | Mannheimia | 0 | 0.000 | 4 | 0.003 |
| 1.01E−01 | genus | Nicoletella | 1 | 0.001 | 5 | 0.004 |
| 6.00E−14 | genus | Pasteurella | 1961 | 1.342 | 355 | 0.279 |
| 6.00E−14 | order | Pseudomonadales | 8016 | 5.485 | 26480 | 20.832 |
| NA | | unclassified | 22 | 0.015 | 25 | 0.020 |
| | | Pseudomonadales | | | | |
| 6.00E−14 | family | Moraxellaceae | 7994 | 5.470 | 26455 | 20.812 |
| NA | | unclassified | 457 | 0.313 | 1999 | 1.573 |
| | | Moraxellaceae | | | | |
| 6.00E−14 | genus | Acinetobacter | 1925 | 1.317 | 1150 | 0.905 |
| 6.00E−14 | genus | Enhydrobacter | 3508 | 2.400 | 15114 | 11.890 |
| 1.09E−40 | genus | Psychrobacter | 1 | 0.001 | 131 | 0.103 |
| 6.00E−14 | genus | Moraxella | 2103 | 1.439 | 8061 | 6.342 |
| 6.00E−14 | order | Xanthomonadales | 4466 | 3.056 | 2757 | 2.169 |
| NA | | unclassified | 2 | 0.001 | 5 | 0.004 |
| | | Xanthomonadales | | | | |
| 2.21E−01 | family | Sinobacteraceae | 0 | 0.000 | 2 | 0.002 |
| NA | | unclassified | 0 | 0.000 | 2 | 0.002 |
| | | Sinobacteraceae | | | | |
| 6.00E−14 | family | Xanthomonadaceae | 4464 | 3.055 | 2750 | 2.163 |
| NA | | unclassified | 1594 | 1.091 | 1089 | 0.857 |
| | | Xanthomonadaceae | | | | |
| 5.74E−07 | genus | Aquimonas | 74 | 0.051 | 21 | 0.017 |
| 2.21E−01 | genus | Arenimonas | 0 | 0.000 | 2 | 0.002 |
| 4.60E−01 | genus | Aspromonas | 0 | 0.000 | 1 | 0.001 |
| 1.28E−02 | genus | Dokdonella | 6 | 0.004 | 17 | 0.013 |
| 1.37E−11 | genus | Dyella | 0 | 0.000 | 34 | 0.027 |
| 4.60E−01 | genus | Frateuria | 0 | 0.000 | 1 | 0.001 |
| 1.39E−02 | genus | Luteimonas | 109 | 0.075 | 69 | 0.054 |
| 6.00E−14 | genus | Lysobacter | 2530 | 1.731 | 808 | 0.636 |
| 4.14E−01 | genus | Pseudoxanthomonas | 6 | 0.004 | 3 | 0.002 |
| 2.44E−02 | genus | Rudaea | 0 | 0.000 | 5 | 0.004 |
| 4.13E−05 | genus | Stenotrophomonas | 19 | 0.013 | 51 | 0.040 |
| 6.00E−14 | genus | Thermomonas | 6 | 0.004 | 631 | 0.496 |
| 6.00E−14 | genus | Xanthomonas | 120 | 0.082 | 17 | 0.013 |
| 4.60E−01 | genus | Xylella | 0 | 0.000 | 1 | 0.001 |
| 6.00E−14 | Phylum | Spirochaetes | 627 | 0.429 | 9370 | 7.371 |
| 6.00E−14 | class | Spirochaetes | 627 | 0.429 | 9370 | 7.371 |
| 6.00E−14 | order | Spirochaetales | 627 | 0.429 | 9370 | 7.371 |
| NA | | unclassified | 1 | 0.001 | 62 | 0.049 |
| | | Spirochaetales | | | | |
| 6.00E−14 | family | Spirochaetaceae | 626 | 0.428 | 9308 | 7.323 |
| NA | | unclassified | 0 | 0.000 | 28 | 0.022 |
| | | Spirochaetaceae | | | | |
| 8.02E−14 | genus | Spirochaeta | 0 | 0.000 | 41 | 0.032 |
| 6.00E−14 | genus | Treponema | 626 | 0.428 | 9239 | 7.268 |
| 6.00E−14 | Phylum | Synergistetes | 168 | 0.115 | 646 | 0.508 |
| 6.00E−14 | class | Synergistia | 168 | 0.115 | 646 | 0.508 |
| 6.00E−14 | order | Synergistales | 168 | 0.115 | 646 | 0.508 |
| 6.00E−14 | family | Synergistaceae | 168 | 0.115 | 646 | 0.508 |
| NA | | unclassified | 111 | 0.076 | 498 | 0.392 |
| | | Synergistaceae | | | | |
| 1.17E−02 | genus | Aminobacterium | 0 | 0.000 | 6 | 0.005 |
| 1.31E−08 | genus | Aminomonas | 42 | 0.029 | 4 | 0.003 |
| 1.93E−02 | genus | Cloacibacillus | 15 | 0.010 | 4 | 0.003 |
| 7.53E−43 | genus | Thermovirga | 0 | 0.000 | 132 | 0.104 |
| 2.21E−01 | genus | Aminiphilus | 0 | 0.000 | 2 | 0.002 |
| 6.00E−14 | Phylum | Tenericutes | 8 | 0.005 | 263 | 0.207 |
| 6.00E−14 | class | Mollicutes | 8 | 0.005 | 263 | 0.207 |
| NA | | unclassified Mollicutes | 1 | 0.001 | 51 | 0.040 |
| 4.66E−46 | order | Acholeplasmatales | 1 | 0.001 | 148 | 0.116 |
| 4.66E−46 | family | Acholeplasmataceae | 1 | 0.001 | 148 | 0.116 |
| 4.66E−46 | genus | Acholeplasma | 1 | 0.001 | 148 | 0.116 |
| 6.00E−13 | order | Mycoplasmatales | 6 | 0.004 | 64 | 0.050 |
| 6.00E−13 | family | Mycoplasmataceae | 6 | 0.004 | 64 | 0.050 |
| 6.00E−13 | genus | Mycoplasma | 6 | 0.004 | 64 | 0.050 |
| 6.00E−14 | Phylum | TM7 | 3194 | 2.186 | 411 | 0.323 |
| 6.00E−14 | genus | TM7_genera_incertae_sedis | 3194 | 2.186 | 411 | 0.323 |
| 8.36E−08 | Phylum | SR1 | 25 | 0.017 | 0 | 0.000 |
| 8.36E−08 | genus | SR1_genera_incertae_sedis | 25 | 0.017 | 0 | 0.000 |
| 9.60E−07 | Phylum | Fusobacteria | 234 | 0.160 | 329 | 0.259 |
| 9.60E−07 | order | Fusobacteriales | 234 | 0.160 | 329 | 0.259 |
| NA | | unclassified | 2 | 0.001 | 1 | 0.001 |
| | | “Fusobacteriales” | | | | |
| 3.40E−07 | family | Fusobacteriaceae | 212 | 0.145 | 309 | 0.243 |
| NA | | unclassified | 8 | 0.005 | 5 | 0.004 |
| | | “Fusobacteriaceae” | | | | |
| 2.23E−01 | genus | Cetobacterium | 6 | 0.004 | 2 | 0.002 |
| 2.15E−08 | genus | Fusobacterium | 196 | 0.134 | 301 | 0.237 |
| 4.60E−01 | genus | Ilyobacter | 0 | 0.000 | 1 | 0.001 |
| 2.82E−01 | genus | Psychrilyobacter | 2 | 0.001 | 0 | 0.000 |
| 9.28E−01 | family | Leptotrichiaceae | 20 | 0.014 | 19 | 0.015 |
| NA | | unclassified | 4 | 0.003 | 3 | 0.002 |
| | | “Leptotrichiaceae” | | | | |
| 2.77E−02 | genus | Leptotrichia | 16 | 0.011 | 5 | 0.004 |
| 5.08E−02 | genus | Sneathia | 0 | 0.000 | 4 | 0.003 |
| 5.61E−03 | genus | Streptobacillus | 0 | 0.000 | 7 | 0.006 |
| 1.74E−06 | Phylum | Chlorobi | 0 | 0.000 | 18 | 0.014 |
| 1.74E−06 | Phylum | Chlorobi | 0 | 0.000 | 18 | 0.014 |
| 1.74E−06 | class | Chlorobia | 0 | 0.000 | 18 | 0.014 |
| 1.74E−06 | order | Chlorobiales | 0 | 0.000 | 18 | 0.014 |
| 1.74E−06 | family | Chlorobiaceae | 0 | 0.000 | 18 | 0.014 |
| NA | | unclassified Chlorobiaceae | 0 | 0.000 | 15 | 0.012 |
| 1.06E−01 | genus | Chloroherpeton | 0 | 0.000 | 3 | 0.002 |
| 1.06E−01 | Phylum | Verrucomicrobia | 0 | 0.000 | 3 | 0.002 |
| 1.06E−01 | class | Opitutae | 0 | 0.000 | 3 | 0.002 |
| NA | | unclassified Opitutae | 0 | 0.000 | 2 | 0.002 |
| 4.60E−01 | order | Puniceicoccales | 0 | 0.000 | 1 | 0.001 |
| 4.60E−01 | family | Puniceicoccaceae | 0 | 0.000 | 1 | 0.001 |
| NA | | unclassified | 0 | 0.000 | 1 | 0.001 |
| | | Puniceicoccaceae | | | | |
| | | Unclassified Bacteria | 2583 | 1.767 | 21668 | 17.046 |
| Total | | | 146140 | 100 | 127113 | 100 |

While comparisons between the PCR amplicon and RT-RNA data derived from the same plaque sample (both classified using the computer-implemented methods of the present invention) revealed similar composition at the phylum level, there were distinct differences between the relative abundances (read density) of sequences for some phyla (FIG. 2). The PCR based approach (amplification dependent method) indicated higher numbers of Actinobacteria, Bacteriodetes, SR1 and TM7 and lower numbers of Proteobacteria and Spirochaetes than PCR-independent methods (FIG. 2). The lower read density of spirochaete sequences obtained by the PCR-based approach (read density of 0.4% and 8.5% for PCR vs. RT-RNA, respectively) is interesting.

General bacterial PCR amplicon inventories of the oral microbiome have previously suggested a low abundance of Spirochaetes, but microscopy studies have demonstrated that between 8 and 54% of oral bacterial cells were Spirochaetes. This underestimation of spirochaete abundance has been attributed to PCR primer bias, and the inventor's data supports this position.

Table 3 below shows the alignment of sequence reads from the 16S rRNA gene PCR amplicon and RT-SSU rRNA datasets classified as belonging to the phylum Spirochaetes:

TABLE 3

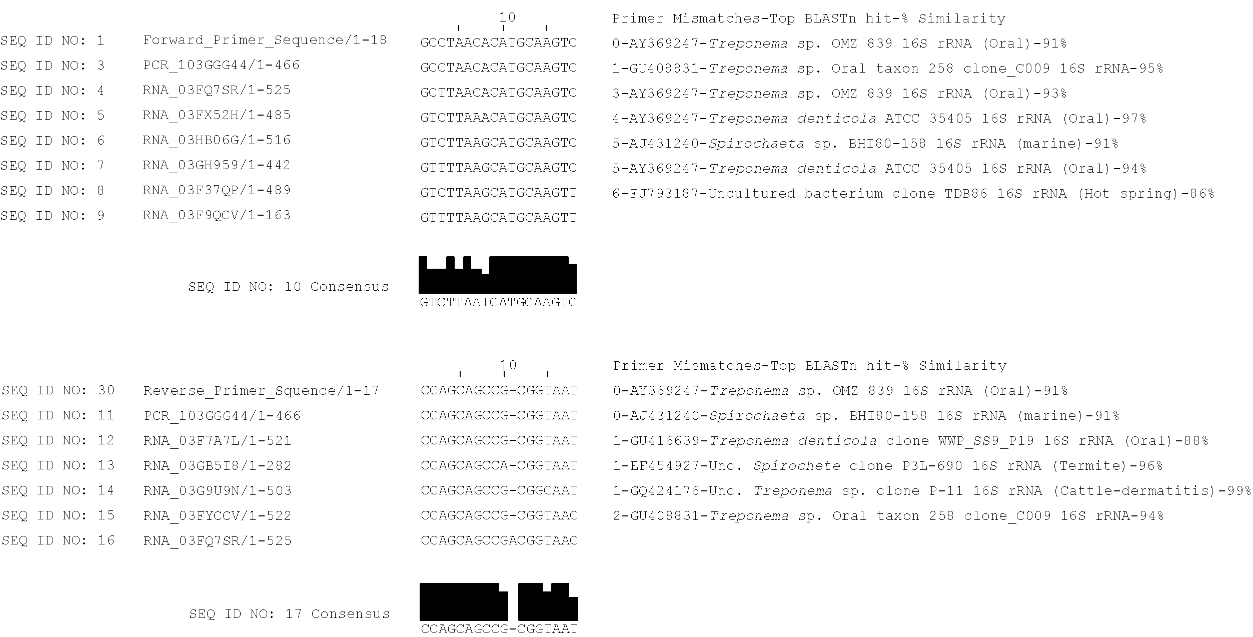

Sequence reads obtained from each dataset were de-replicated using CD-HIT (http://weizhong-lab.ucsd.edu/cd-hit/) and representative sequences for each OTU group aligned against ‘good’ quality reference Spirochaete sequences from the Ribosomal Database Project website (http://rdp.cme.msu.edu/) to produce Table 3. In Table 3, left column, sequence names beginning with PCR are from the PCR amplicon dataset and sequences beginning with RNA are from the RT-SSU rRNA dataset. The column to the right of the alignment in Table 3 highlights the number of mismatches between the sequence group and the primer site, followed by the Genbank accession number of the closest BLASTn match to that group. The ecological source of the closest reference sequence is presented in parentheses where not stated in the BLAST description and the % similarity to our query sequence is also shown. The sequence of the forward and reverse primers used to create the PCR amplicon library (V1-V3F 5′-GCCTAACACATGCAAGTC-3′ SEQ ID NO: 1 and the reverse complement of V1-V3R 5′-ATTACCGCGGCTGCTGG-3′ SEQ ID NO: 2) are shown as the top sequence in each alignment.

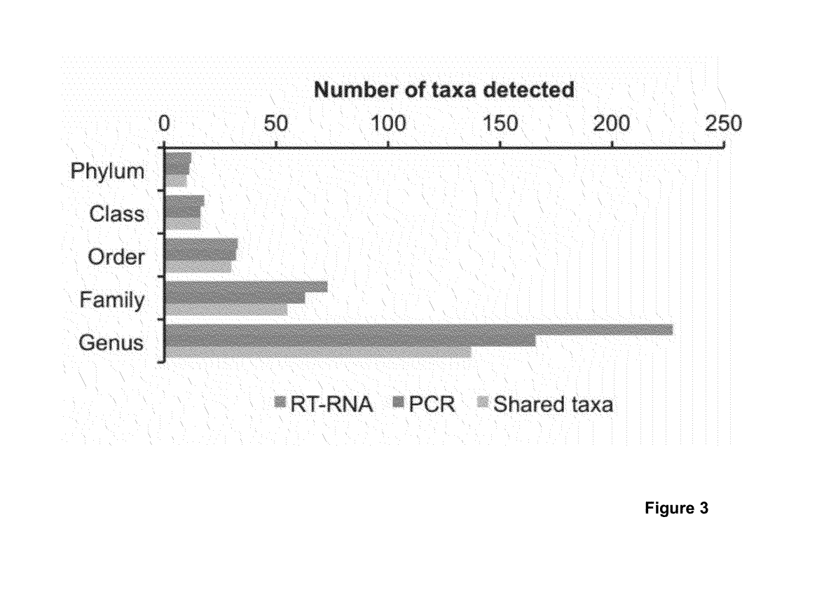

The differences in the number of taxa detected at each taxonomic rank by both the amplification-independent RT-SSU rRNA method of the present invention and the PCR based approach (amplification-dependent method) is shown in FIG. 3. The amplification-independent RT-SSU rRNA method of the present invention consistently detected more bacterial taxa at every taxonomic level. In fact, at the genus-level 40% more diversity was detected in the RT-SSU rRNA dataset. There are several instances where taxa are found in one library and not the other. Where sequences are found only in the PCR amplicon library, they are usually rare and never comprise more than 0.02% of the bacterial population; whereas sequences unique to the RT-SSU rRNA library were found in higher percentages, e.g. Chryseobacterium, Diaphorobacter, Pseudorhodoferax, Thermovirga, and members of the Oxalobacteraceae comprised 0.05% to <0.9% of the total bacterial sequence counts while Variovorax and Aquaspirillum comprised 1.1 and 1.2% of the total bacterial sequence counts, respectively. Furthermore, many of the sequences could only be resolved above the genus level, suggesting the presence of potentially novel taxa at every phylogenetic rank.

Due to the randomly fragmented nature of the RT-RNA reads, it is possible that some sequences may cover conserved areas of the 16S rRNA gene and are therefore less phylogenetically informative than reads containing variable regions. However, the computer-implemented methods of the present invention screen sequence reads against user-defined variable regions of the 16S rRNA gene to improve the phylogenetic resolution of the reads.

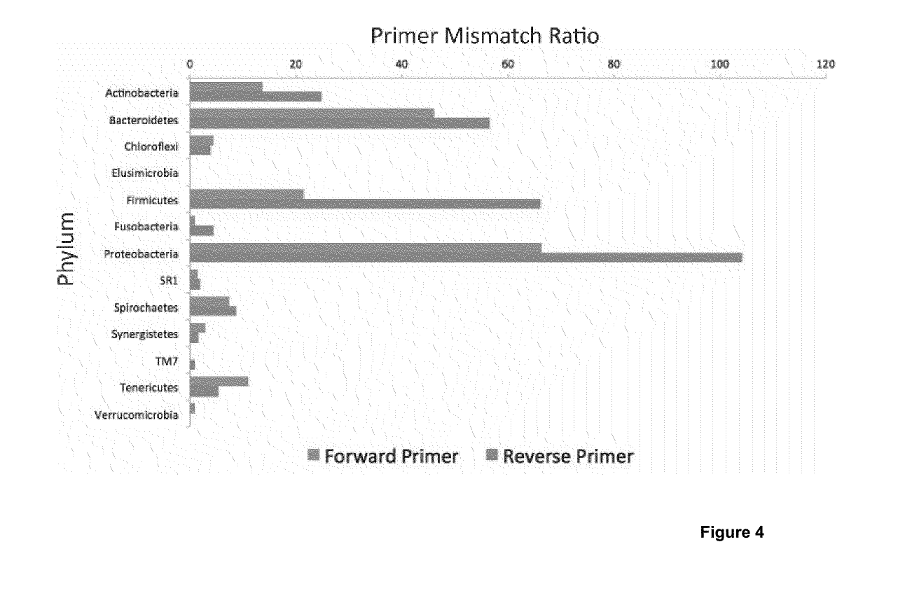

One explanation for the increased taxonomic diversity observed in the RT-SSU rRNA dataset is that PCR primers only target ‘known’ diversity. To investigate this, the inventors aligned the RT-SSU rRNA query sequences with their closest database match and identified insertions, deletions and mutations present within the regions of the SSU rRNA reads that correspond to the PCR primer binding sites of the primers used to amplify the 16S rRNA gene for the amplicon library. Sequence mismatches with at least one of the primers used to generate the 16S rRNA gene amplicon library were detected in all phyla observed in the RT-SSU rRNA library, with the exception of the phylum Elusimicrobia (FIG. 4). Previously undetected sequence diversity within the binding site of the general bacterial primer set used is therefore one explanation for the increased diversity and different relative abundances observed in the RT-SSU rRNA dataset compared to 16S rRNA gene amplicons from the same sample. This supports the notion that novel centres of variation are detected via the amplification-independent methods of the present invention.

### Example 4

### qPCR Analysis of ‘Artificial’ Microbial Communities after PCR Amplification of the 16S rRNA Gene

Primer mismatches do not explain all of the differences found in the relative abundance discrepancies between the datasets for the amplification-dependent methods and the amplification-independent methods of the present invention.

To investigate the effect of PCR amplification bias, the inventors generated ‘artificial’ microbial community comprising an artificial mixture of five cloned 16S rRNA genes, each possessing the universal primer binding sites used to produce the PCR amplicon library. Canine oral bacterial taxa that were identified as under-represented (Fusobacterium and Proteobacteria-Desulphomicrobium) or over-represented (Actinobacteria and Proteobacteria-Cardiobacterium) in the PCR amplicon dataset were used and one that had a similar abundance (Treponema) in the PCR and RT-SSU rRNA datasets. The members of the artificial community were mixed in known ratios of gene copy number and subjected to 10, 20 or 30 cycles of PCR. Subsequently, primer sets specific for each member of the artificial community were used to quantify the abundance of each 16S rRNA gene in the resulting amplicon pool by qPCR via a direct quantification strategy using taxon-specific standards.

Plasmid DNA was extracted using a QIAprep Spin Midiprep kit (Qiagen, West Sussex), quantified using a Qubit fluorimeter, and linearised using Hind III, which cuts the plasmid in one location and did not cut the 16S rRNA gene insert. Linearised plasmids were purified using a QIAquick PCR purification kit (Qiagen), quantified using a Qubit fluorimeter, and the copy number for each plasmid preparation was subsequently determined. Purity of the DNA was assessed using a Nanodrop.

Prior to PCR, linearised plasmid DNA derived from each of the five 16S rRNA gene clones was combined in different quantities to simulate an ‘artificial’ canine oral microbial community, so that some sequences were more abundant than others. The final ratio of the five clone mixture (A9, C10, F10, E3 and E9) was 1:3:8:2:10 respectively, as determined by qPCR. The ‘artificial’ microbial community was subjected to PCR amplification via PCR using the same V1-3 16S rRNA gene-specific primers used to generate the canine oral 16S rRNA gene PCR amplicon library in this study (63f 5′-GCCTAACACATGCAAGTC-3′ (SEQ ID NO: 18) and 518r 5′-ATTACCGCGGCTGCTGG-3′ (SEQ ID NO: 19) universal primers V1-V3 forward and reverse. PCR conditions: 94° C. (4 minutes), 94° C. (30 seconds×number of cycles), 56° C. (30 seconds×number of cycles), 72° C. (30 seconds×number of cycles), 72 (10 minutes), hold at 4° C. DNA template (14) was added to 49 μL of mastermix comprising of 22 μL DEPC, 0.2 mM of each of the forward and reverse V1-V3 primers and 25 μL of Biomix Red obtained from Bioline (London). To test the effect of cycle number on the final ratios, 3 separate PCR experiments were performed, each with varying rounds of amplification (10 cycles, 20 cycles and 30 cycles). Each PCR reaction was conducted in triplicate.

To quantify the abundance of each cloned 16S rRNA gene sequence in the PCR amplicon mix produced by 10, 20 and 30 cycles of PCR with general bacterial primers, genus specific primers specific to each clone were designed using sequence alignments to locate regions of variability.

In particular, primer sets were designed for use in qPCR experiments in order to assess the change in ratio of 16S rRNA gene copies resulting from amplification of an initial artificial 5-member microbial community subjected to 10, 20 and 30 cycles of PCR with the general bacterial primer set V1-V3F 5′-GCCTAACACATGCAAGTC-3′ (SEQ ID NO:1) and V1-V3R 5′-ATTACCGCGGCTGCTGG-3′ (SEQ ID NO:2):

TABLE 4
Genus-specific 16S rRNA gene PCR primer sets

| Primer | Specificity (genus) | Sequence |
|---|---|---|
| E3 F | Cardiobacterium | 5′-GCAGCACGAGAAAGC-3′ |
| | | SEQ ID NO: 20 |
| E3 R | Cardiobacterium | 5′-ATCAGCGCGAGGTCT-3′ |
| | | SEQ ID NO: 21 |
| E9 F | Fusobacterium | 5′-CTCTTAGACCGGGAC-3′ |
| | | SEQ ID NO: 22 |
| E9 R | Fusobacterium | 5′-GGGACGCAAAGCTCT-3′ |
| | | SEQ ID NO: 23 |
| A9 F | Actinomycetaceae | 5′-ACGGGATCTGATGGG-3′ |
| | | SEQ ID NO: 24 |
| A9 R | Actinomycetaceae | 5′-CCCACAACCACCATG-3′ |
| | | SEQ ID NO: 25 |
| C10 F | Treponema | 5′-CGGCAAGAGAGAAGCTT-3′ |
| | | SEQ ID NO: 26 |
| C10 R | Treponema | 5′-CTCTAACAGATGCGGTC-3′ |
| | | SEQ ID NO: 27 |
| F10 F | Desulfomicrobium | 5′-CCGGGAATGAGTAGAGT-3′ |
| | | SEQ ID NO: 28 |
| F10 R | Desulfomicrobium | 5′-CATCCTTTACCGACTCC-3′ |
| | | SEQ ID NO: 29 |

For the generation of standard curves for the absolute quantification of plasmid copy number, linearised and purified plasmids were diluted by six 10-fold serial dilutions, representing 10⁸-10³ 16 S rRNA gene copies. Each dilution in the standard curve was assayed in triplicate. Five μL of the ‘artificial’ mixed microbial community was combined with 45 μL of mastermix containing 19 μL DEPC H₂O, 0.54 forward primer, 0.5 μL reverse primer and 254 Sensimix SYBR Green No ROX (×2) obtained from Bioline (London). The reaction was optimized for each clone in order to find the melting temperature (Tm), extension time and primer concentration that would give the highest efficiency percentage and an R² value close to 1. For each standard curve a non-template control (NTC) was also run alongside the serial dilutions, in order to check for non-specific amplification.

To quantify the post-PCR abundance of each 16S rRNA gene sequence, genus specific primers were used in qPCR assays in conjunction with clone-specific standard curves for the absolute quantification of gene copy number of each 16S rRNA gene sequence in the artificial microbial community. Amplicon mixtures derived from the artificial community after 10, 20 and 30 cycles of PCR were diluted to appropriate levels so that the obtained Ct values would fall within the range of the standard curves and added to qPCR assays for quantification of each 16S rRNA sequence type as described above.

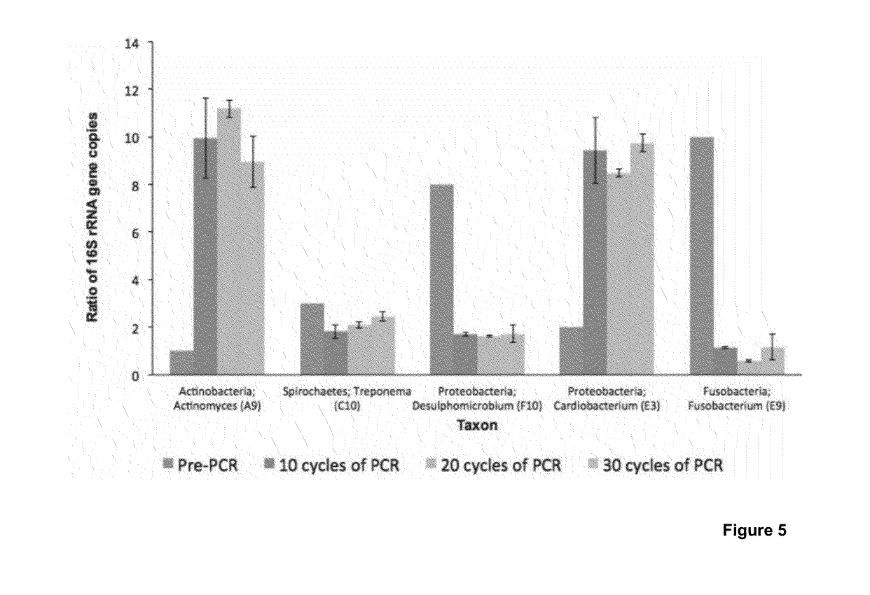

These data demonstrate significant differences in the amplification efficiencies of each 16S rRNA gene (FIG. 5); the Actinobacteria 16S rRNA gene was over-represented (Pre-PCR ratio of 16S gene copies=1, post-PCR average ratio of 16S rRNA gene copies=10), whereas the Fusobacterium 16S rRNA gene was under-represented in the amplified gene pool (Pre-PCR ratio of 16S gene copies=10, post-PCR average ratio of 16S rRNA gene copies=1). These observations are consistent with previous findings concerning the canine oral microbiome in which members of various phyla are represented in varying relative abundances from the same biological sample depending on which set of ‘universal’ bacterial primer sets was used, e.g. F24+AD35/C72 [9-27F/1492-1509R], F24/Y36 [9-29F/1525-1241R]. Two of the cloned 16S rRNA genes derived from separate genera of the Proteobacteria gave contrasting amplification efficiencies (FIG. 5; Desulphomicrobium Pre-PCR ratio of 16S gene copies=8, post-PCR average ratio of 16S rRNA gene copies=2, Cardiobacterium Pre-PCR ratio of 16S gene copies=2, post-PCR average ratio of 16S rRNA gene copies=9); the Spirochaetes clone displayed a similar abundance to the actual ratio of gene copies in the artificial community (FIG. 5). The cloned 16S rRNA gene sequences could all be amplified by the universal bacterial primers (V1-V3F (16) and V1-V3R (17)), and though there were a few mismatches with the V1-V3F primer, the last 11 nucleotides matched perfectly. Furthermore the comparative amplification efficiencies of each cloned template did not correlate with universal primer mismatches in the clone sequence templates. Consequently, amplification efficiencies are not controlled merely by primer recognition strength, or % GC content, but by other as yet uncharacterised properties, possibly inherent to the DNA template, e.g. secondary structure.

The above Examples suggest that the failure to detect certain taxa via amplification-dependent sample preparation approaches is a combination of several factors that include primer mismatches, differential PCR amplification efficiencies and potentially, other phenomena reported elsewhere.

The novel amplification-independent methods of the present invention and the novel computer-implemented methods of the present invention allow simultaneous determination of microbial diversity and SSU rRNA relative abundance within the same sample.

Additionally, the novel computer-implemented methods of the present invention can usefully be employed with existing amplification-dependent technologies to improve the speed and accuracy of sequence classification.

In fact, the novel computer-implemented methods of the present invention can be usefully employed to assist in the quick and accurate classification of any isolated biological sample containing DNA, RNA or protein.

### Example 5

### Isolation and Preservation of Biological Samples

A key consideration, often overlooked in oil and gas industry sampling, is the efficiency of extraction. Unless there is a linear relationship between extraction input and yield, over the very wide potential ranges of DNA/RNA concentrations typically sampled from real-life assets, then any resulting qPCR and RT-qPCR data based on these extracts can be incorrect.

The table below shows the comparison between the prior art methodologies (bulk water and filter-based methods) and the present invention:

| | | Total RNA | Altered Composition |
|---|---|---|---|
| Sampling Method | [Total RNA] | as 23S | and Gene Expression? |
| Bulk Water | 274 ng/μl | 1% | Yes (prolonged wholly |
| | | | unnatural environment) |
| Filter-Based Kit | 11 ng/μl | 0% | Yes (designed to work |
| | | | with mammalian cells) |
| Present invention | 954 ng/μl | 11% | NO (rapid cell lyis/ |
| | | | chemical preservation) |

For each methodology, sample processing took place on equal numbers of living microbial cells in produced water. All three processed samples were then left in situ at room temperature for a week to mimic transportation from platform/asset to laboratory.

The present invention provides a higher total RNA yield than the prior art methodologies, the highest proportion of high molecular weight RNA (23S) and also preserved the microbial community composition (genomic DNA) and gene expression patterns (mRNA) at the point-of-capture ready for downstream molecular biology analyses. In particular, no altered composition or gene expression was observed with the present invention, with the RNA preserved for at least a week.

FIG. 7 shows input-versus-output over orders-of-magnitude differences in prokaryotic genomic DNA recovery from an oil and gas industry low biomass environmental sample—using the sample isolation methods of the present invention. There is a clear linear relationship, from input values likely to be higher than those encountered in real-life scenarios, right down the extremely low inputs commonly resulting from low biomass environments.

### REFERENCES

1. Olsen G J, Lane D J, Giovannoni S J, Pace N R, & Stahl D A (1986) Microbial ecology and evolution—a Ribosomal-RNA Approach. Ann. Rev. Microbiol. 40:337-365.
2. Pace N R (1997) A molecular view of microbial diversity and the biosphere. Science 276(5313):734-740.
3. Ward D M, Weller R, & Bateson M M (1990) 16S ribosomal-RNA sequences reveal numerous uncultured microorganisms in a natural community. Nature 345(6270):63-65.
4. Woese C R & Fox G E (1977) Phylogenetic structure of prokaryotic domain-primary kingdoms. PNAS USA 74(11):5088-5090.
5. Staley J T & Konopka A (1985) Measurement of in situ activities of nonphotosynthetic microorganisms in aquatic and terrestrial habitats. Ann. Rev. Microbiol. 39:321-346.
6. Fox J L (2005) Ribosomal gene milestone met, already left in dust. ASM News 71(1):6-7.
Polz M F & Cavanaugh C M (1998) Bias in template-to-product ratios in multitemplate PCR. Appl. Environ. Microbiol. 64(10):3724-3730.
8. Shakya M, et al. (2013) Comparative metagenomic and rRNA microbial diversity characterization using archaeal and bacterial synthetic communities. Environ. Microbiol. 15(6):1882-1899.
9. Suzuki M T & Giovannoni S J (1996) Bias caused by template annealing in the amplification of mixtures of 16S rRNA genes by PCR. Appl. Environ. Microbiol. 62(2):625-630.
10. von Wintzingerode F, Gobel U B, & Stackebrandt E (1997) Determination of microbial diversity in environmental samples: pitfalls of PCR-based rRNA analysis. FEMS Microbiol. Rev. 21(3):213-229.
11. Hong S H, Bunge J, Leslin C, Jeon S, & Epstein S S (2009) Polymerase chain reaction primers miss half of rRNA microbial diversity. ISME 3(12):1365-1373.
12. Jeon S, et al. (2008) Environmental rRNA inventories miss over half of protistan diversity. BMC Microbiol. 8.
13. Lanzen A, et al. (2011) Exploring the composition and diversity of microbial communities at the Jan Mayen hydrothermal vent field using RNA and DNA. FEMS Microbiol. Eco. 77(3):577-589.
14. Urich T, et al. (2008) Simultaneous assessment of soil microbial community structure and function through analysis of the meta-transcriptome. PLOS One 3(6):e2527.
15. Blazewicz S J, Barnard R L, Daly R A, & Firestone M K (2013) Evaluating rRNA as an indicator of microbial activity in environmental communities: limitations and uses. ISME J.
16. Marchesi J R, et al. (1998) Design and evaluation of useful bacterium-specific PCR primers that amplify genes coding for bacterial 16S rRNA. Appl. Environ. Microbiol. 64(2):795-799.
17. Muyzer G, Dewaal E C, & Uitterlinden A G (1993) Profiling of complex microbial-populations by denaturing gradient gel-electrophoresis analysis of polymerase chain reaction-amplified genes-coding for 16S ribosomal-RNA. Appl. Environ. Microbiol. 59(3):695-700.
18. Tringe S G & Hugenholtz P (2008) A renaissance for the pioneering 16S rRNA gene. Curr. Opin. Microbiol. 11(5):442-446.
19. Dewhirst F E, et al. (2012) The canine oal microbiome. (Translated from English) PLOS One 7(4).
20. Wade W G (2013) The oral microbiome in health and disease. Pharmacol. Res. 69(1):137-143.
21. Lepp P W, et al. (2004) Methanogenic Archaea and human periodontal disease. PNAS USA 101(16):6176-6181.
22. Ghannoum M A, et al. (2010) Characterization of the oral fungal microbiome (mycobiome) in healthy individuals. PLOS Pathog. 6(1).
23. Quast C, et al. (2013) The SILVA ribosomal RNA gene database project: improved data processing and web-based tools. Nucleic Acids Res. 41(D1):D590-D596.
24. Caporaso J G, et al. (2010) QIIME allows analysis of high-throughput community sequencing data. Nature Meth. 7(5):335-336.
25. Wang Q, Garrity G M, Tiedje J M, & Cole J R (2007) Naïve Bayesian classifier for rapid assignment of rRNA sequences into the new bacterial taxonomy. Appl. Environ. Microbiol. 73(16):5261-5267.
26. Choi B K, Paster B J, Dewhirst F E, & Gobel U B (1994) Diversity of cultivable and uncultivable oral spirochetes from a patient with severe destructive periodontitis. Infect. Immun. 62(5):1889-1895.
27. Loesche W J (1988) The role of spirochetes in periodontal disease. Adv. Dent. Res. 2(2):275-283.
28. Engelbrektson A, et al. (2010) Experimental factors affecting PCR-based estimates of microbial species richness and evenness. ISME 4(5):642-647.
29. Wu J Y, et al. (2010) Effects of polymerase, template dilution and cycle number on PCR based 16S rRNA diversity analysis using the deep sequencing method. BMC Microbiol. 10.
30. Griffiths R I, Whiteley A S, O'Donnell A G, & Bailey M J (2000) Rapid method for coextraction of DNA and RNA from natural environments for analysis of ribosomal DNA- and rRNA-based microbial community composition. Appl. Environ. Microbiol. 66(12):5488-5491.
31. Cole J R, et al. (2005) The Ribosomal Database Project (RDP-II): sequences and tools for high-throughput rRNA analysis. Nucleic Acids Res. 33:D294-D296.
32. McDonald D, et al. (2012) An improved Greengenes taxonomy with explicit ranks for ecological and evolutionary analyses of bacteria and archaea. ISME J. 6(3):610-618.

1-110. (canceled)

111. A method for generating a signature map for sequence classification of a biological sample, the method comprising:
(i) obtaining a nucleic acid from a biological sample;
(ii) sequencing the nucleic acid to obtain a sequence;
(iii) associating the sequence with a sequence identifier (ID), wherein the sequence a comprises plurality of groups of k-mers, and each group of k-mers defines a node in a multilevel hierarchy which defines a relationship between the groups of k-mers;
(iv) associating each group of k-mers with a respective group identifier (ID), and determining a frequency of the k-mers in each group;
(v) generating a group signature array for each group of k-mers, wherein each group signature array comprises the k-mers in each group that have the highest frequency relative to that group;
(vi) generating a signature map comprising each group signature array and at least one of the sequence identifier (ID) or the group identifier (ID); and
(vii) outputting the signature map to be used to classify the sequence.

112. The method of claim 111, wherein the nucleic acid is DNA.

113. The method of claim 111, wherein the nucleic acid is RNA.

114. The method of claim 113, wherein the RNA is 16s RNA.

115. The method of claim 113, wherein the RNA is Small Sub-Unit ribosomal RNA (SSU rRNA).

116. The method of claim 113, wherein the SSU rRNA is isolated and purified using a composition comprising a ribonuclease inhibitor and a deoxyribonuclease to remove DNA from the sample.

117. The method of claim 113, wherein the SSU rRNA is reverse transcribed into ds cDNA.

118. The method of claim 113, wherein the reverse transcription is performed using random primers for the SSU rRNA.

119. The method of claim 111, wherein the method further comprises amplifying the nucleic acid prior to sequencing.

120. The method of claim 111, wherein the method does not comprises amplification of the nucleic acid prior to sequencing.

121. The method of claim 111, wherein the biological sample is from an oil well.

122. The method of claim 111, wherein the biological sample is preserved using a chaotropic agent.

123. The method of claim 122, wherein the biological sample is stored at ambient temperatures.

124. The method of claim 122, wherein the method further comprises:
i) subjecting the biological sample to microbial cell lysis;
ii) contacting the lysed biological sample with a slurry of size-selected silicon dioxide to form a nucleic acid-silicon dioxide complex;
iii) isolating the nucleic acid-silicon dioxide complex; and
iv) sequencing the nucleic acid-silicon dioxide complex.

125. The method of claim 122, further comprising subjecting the biological sample to an activated charcoal treatment step.

126. The method of claim 111, wherein steps (iii)-(vii) are performed by a computer.

127. The method of claim 111, wherein the method further comprises converting a value of each group into a string and storing the string for each group with the respective group identifier.

128. The method of claim 111, wherein when more than three sequences are associated with a group, the method comprises clustering the sequences into one or more sub-groups, each with a respective sub-group identifier.

129. The method of claim 111, wherein the generation of the group signature array comprises depth first recursive processing of the groups in the hierarchy.

130. The method of claim 129, wherein the depth first recursive processing comprises processing a parent group and each child group of the parent group by scaling each child group signature array by a maximum value (N), and adding the scaled child group signature array to the parent group signature array.

131. The method of claim 130, wherein the method further comprises converting the sequences in the child group to the same signature array format as the parent group signature array to generate a child sum array for each child, and adding the converted sequences to one another to form a children sum array.

132. The method of claim 130, wherein the method further comprises generating a signature group array for each child by:
(i) subtracting the child sum array from the children sum array to produce a sibling sum array;
(ii) filling the group signature array with the child k-mers in each group with a higher frequency than k-mers in at least one sibling group up to a predetermined frequency value; and
(iii) scaling the group signature array by the maximum value (N).

133. The method of claim 130, wherein the method further comprises classifying the sequence by comparing the sequence to a first child group signature array and comparing the sequence to at least one other child group signature array until no better match can be identified between the sequence and a child group signature array.

134. The method of claim 111, wherein the method further comprises clustering sequences with a similarity above a predetermined level and mapping each cluster of sequences to the signature map.
